(19) United States
(12) Patent Application Publication
(10) Pub. No.: US 2017/0283874 A1
(43) Pub. Date: Oct. 5, 2017

(54) Analytical Methods and Arrays for Use in the Same

(71) Applicant: SENZAGEN AB, Lund (SE)

(72) Inventors: Malin Lindstedt, Bunkeflostrand (SE); Carl Borrebaeck, Lund (SE); Henrik Johansson, Malmo (SE); Ann-Sofie Albrekt, Teckomatorp (SE)

(21) Appl. No.: 15/624,955

(22) Filed: Jun. 16, 2017

Related U.S. Application Data
(63) Continuation of application No. 14/396,422, filed on Oct. 23, 2014.

(30) Foreign Application Priority Data
Apr. 26, 2012 (GB) 1207297.1

(51) Int. Cl. C12Q 1/68; G01N 33/50; G01N 33/68

(52) U.S. Cl. CPC C12Q 1/6883; G01N 33/6893; C12Q 2600/106; G01N 2800/52; G01N 33/5023; G01N 2800/24; G01N 33/5047; C12Q 2600/158

(57) ABSTRACT

The present invention relates to an in vitro method for identifying agents capable of inducing respiratory sensitization in a mammal and arrays and diagnostic kits for use in such methods. In particular, the methods include measurement of the expression of the biomarkers listed in Table 1A, Table 1B and/or Table 1C in MUTZ-3 cells exposed to a test agent.

This application is a continuation application of U.S. patent application Ser. No. 14/396,422, filed Oct. 23, 2014, which is a §371 application of PCT/IB2013/053321, filed Apr. 26, 2013, which claims priority to GB 1207297.1, filed on Apr. 26, 2012. The foregoing applications are incorporated by reference herein.

### FIELD OF THE INVENTION

The present invention relates to a method for identifying agents capable of inducing respiratory sensitization and arrays and analytical kits for use in such methods.

### BACKGROUND

Allergy, in general, is defined as an adverse condition which is manifested following an immune response to an otherwise innocuous antigen. It is a member of a class of outcomes termed hypersensitivity reactions which are defined as harmful immune responses which result in tissue injury (Janeway, C., Travers, P., Hunt, S., Walport, M., 1997. Allergy and hypersensitivity. ImmunoBiology: The Immune System in Health and Disease. Garland Publishing, New York). The resulting conditions that are of particular concern to industrial toxicologists include both respiratory allergy and allergic contact dermatitis (ACD). Respiratory allergy is a hypersensitivity reaction of the upper and/or lower respiratory tract to a xenobiotic. This hypersensitivity reaction is immediate, with clinical characteristics occurring within minutes to hours after xenobiotic exposure, and can include wheezing, breathlessness, tightness in the chest, bronchoconstriction, and/or nasal congestion. In extreme cases the reaction can elicit hypotension and life-threatening anaphylaxis. In the general population, respiratory allergy is most frequently induced by environmental proteins including pollen, dust mite excreta and animal dander. However, in occupational settings, respiratory allergy can be mediated by industrial compounds including high molecular weight (HMW) compounds, such as protein detergents, and low molecular weight (LMW) chemicals. Due to their small size, LMW chemical allergens act as haptens which first react with proteins to create a complex that is then able to initiate an immune response.

Development of respiratory allergy to HMW and LMW compounds can contribute to the development of occupational asthma which is characterized by variable airflow limitation and/or non-specific bronchial hyper-responsiveness due to causes and conditions attributable to a specific work environment (Chan-Yeung, M., Malo, J. L., 1994. Aetiological agents in occupational asthma. Eur. Respir. J. 7, 346-371; Karol, M. H., 1994. Animal models of occupational asthma. Eur. Respir. J. 7, 555-568). It is important to note that in addition to this immunological etiology, non-immunogenic agents such as irritants also play a significant role in the development of occupational asthma. In many cases, concurrent exposure to both allergens and irritants contributes to the condition. Clinical investigations have suggested that up to 20% of adult onset asthma is caused by occupational factors and that 90% of these cases involve an immunological mechanism (Mapp, C. E., 2005. Genetics and the occupational environment. Curr. Opin. Allergy Clin. Immunol. 5, 113-118). Furthermore, occupational asthma is the most prevalent occupational lung disease in developed countries. As a result, identification and characterization of compounds which have the potential to act as respiratory allergens are an important area of research for industrial toxicologists.

Not all compounds that provoke a specific immune response will have the potential to cause hypersensitivity of the respiratory tract. A larger number of compounds are associated with skin hypersensitivity and the development of ACD and are believed to have no sensitizing effect on the respiratory tract (Kimber, I., Dearman, R. J., 2005. What makes a chemical a respiratory sensitizer? Curr. Opin. Allergy Clin. Immunol. 5, 119-124). Unlike respiratory allergy, ACD is an example of a delayed-type hypersensitivity reaction resulting from cell-mediated immune responses (Janeway et al., 1997 supra). ACD is one of the most common occupational diseases with a number of compounds being implicated as causative agents, therefore, proactive identification and characterization of these compounds are also of considerable importance (Saary, J., Qureshi, R., Palda, V., DeKoven, J., Pratt, M., Skotnicki-Grant, S., Holness, L., 2005. A systematic review of contact dermatitis treatment and prevention. J. Am. Acad. Dermatol. 53, 845).

The development of hypersensitivity resulting in respiratory allergy or ACD consists of two distinct stages. The first is sensitization, which involves the development of an immune status, while the second is elicitation, which results in the clinical manifestation of allergy (Briatico-Vangosa, G., Braun, C. L., Cookman, G., Hofmann, T., Kimber, I., Loveless, S. E., Morrow, T., Pauluhn, J., Sorensen, T., Niessen, H. J., 1994. Respiratory allergy: hazard identification and risk assessment. Fundam. Appl. Toxicol. 23, 145-158). As a result, previously unexposed (naive) but susceptible individuals do not experience allergic symptoms the first time they are exposed to an allergenic protein or chemical. At a minimum it requires two exposures; however, in many cases it may require repeated exposures over weeks or months. During the initial encounters of a susceptible individual to an allergic compound, the compound is recognized as foreign by dendritic cells (antigen processing and presenting cells), presented to T cells, and a specific primary immune response is provoked which results in sensitization. This can be followed by the actual elicitation of allergy upon subsequent exposure of the sensitized individual to the same compound. Elicitation is mediated through the activation of an immune response and the resultant cellular signals which result in an inflammatory reaction and symptoms of the allergy. The nature and severity of the allergic reaction are dependent upon a number of factors including the genetic background of the individual, the characteristics of the allergen, as well as the route, duration and intensity of the exposure during both the sensitization and elicitation stages (Arts, J. H., Kuper, C., 2003. Approaches to induce and elicit respiratory allergy: impact of route and intensity of exposure. Toxicol. Lett. 140-141, 213-222; Arts, J. H., Mommers, C., de Heer, C., 2006. Dose—response relationships and threshold levels in skin and respiratory allergy. Crit. Rev. Toxicol. 36, 219-251).

Despite some general similarities, there are important mechanistic differences in the currently understood etiology of respiratory allergy and ACD. Generally, respiratory allergy is classified as a type I hypersensitivity reaction involving IgE while ACD is a type IV hypersensitivity reaction which is mediated by T cells (Janeway et al., 1997 supra). These hypersensitivities are thought to develop according to specific mechanisms that depend on the differential activation of functional subpopulations of T helper (Th) cells, namely, Th1 and Th2 cells. Development of respiratory sensitization and allergy has been associated with the preferential induction of a Th2 population of T lymphocytes. Th2 cells are characterized by the production of high amounts of interleukins (IL)-4, -10 and -13. The production of these cytokines favours humoral immune function and the stimulation and differentiation of B cells to produce IgE (reviewed in Dearman, R. J., Betts, C. J., Humphreys, N., Flanagan, B. F., Gilmour, N. J., Basketter, D. A., Kimber, I., 2003. Chemical allergy: considerations for the practical application of cytokine profiling. Toxicol. Sci. 71, 137-145). These antibodies bind to receptors on the surface of mast cells and basophils. Upon subsequent exposure to the allergen, these cells release various inflammatory mediators including histamine, leukotrienes and cytokines, which results in the immediate hypersensitivity of respiratory allergy. In addition to promoting IgE production, Th2 cytokines also promote the growth and differentiation of other cells involved in respiratory allergy including mast cells and eosinophils (reviewed in Kimber, I., 1996. Chemical-induced hypersensitivity. In: Smialowicz, R. J., Holsapple, M. P. (Eds.), Experimental Immunotoxicology. CRC Press, New York, pp. 391-417). Upon repeated exposure to allergenic compounds and the elicitation of respiratory allergy, extensive airway remodeling, mucus accumulation and chronic inflammatory responses may develop which contribute to the development of an asthmatic condition.

In contrast, the development of contact sensitization and ACD has been primarily associated with the induction of a Th1 population of T lymphocytes. These cells are characterized by the production of IL-2, interferon-gamma (IFN-γ) and tumor necrosis factor-β (TNF-β). Research has shown that the development of delayed contact hypersensitivity is specifically dependent on Th1 cells and the production of IFN-β (Diamantstein, T., Eckert, R., Volk, H. D., Kupier-Weglinski, J. W., 1988. Reversal by interferon-gamma of inhibition of delayed-type hypersensitivity induction by anti-CD4 or anti-interleukin 2 receptor (CD25) monoclonal antibodies. Evidence for the physiological role of the CD4+ TH1+ subset in mice. Eur. J. Immunol. 18, 2101-2103). The sensitization response is associated with the generation of memory T cells which are activated upon subsequent encounter with the antigen resulting in the hypersensitivity response. This reaction involves the activation of keratinocytes and the release of proinflammatory cytokines to recruit non-antigen specific T cells and monocytes to the site of contact which results in an acute inflammatory response. Interestingly, IFN-γ produced by Th1 cells also antagonizes Th2 cell responses and the production of IgE, while IL-4 produced by Th2 cells antagonizes the development of Th1 cells. Furthermore, IFN-γ has been found to inhibit mast cell function in respiratory allergy, while IL-4 depresses the elicitation stage of ACD (reviewed in Kimber, 1996 supra). Therefore, not only do the cytokines of each Th cell type promote the growth and differentiation of their lineage and the subsequent hypersensitivity response, they also antagonize the proliferation of the other cell population as a means of further directing the immune response.

The above distinction between respiratory allergy and ACD is of considerable importance from a hazard assessment and regulatory perspective. Researchers have explored a number of animal models and experimental approaches to identify compounds with the potential to cause respiratory allergy, however, none of the approaches are widely applied or fully accepted by the research community or regulatory agencies (Arts et al., 2006 supra.). In contrast, there are a number of guideline assays for the detection of compounds with the potential to cause contact sensitization and ACD. Given the general similarity of the sensitization response in respiratory allergy and ACD, it has been suggested that models for identifying the potential for contact sensitization could also be used for the assessment of the potential for respiratory allergy. However, due to the known mechanistic differences and the more serious health and regulatory implications for classification as a respiratory allergen, the accurate identification of these compounds and their distinction from compounds inducing ACD is critical. What is required is a consistent, systematic and accepted approach for assessing the respiratory allergy potential of both protein and chemical compounds.

Respiratory allergy is a type I hypersensitivity reaction of the upper and lower respiratory tract to xenobiotic proteins or chemicals, with clinical symptoms typically including wheezing, breathlessness, bronchochonstriction and asthmatic attacks (Boverhof D R, Billington R, Gollapudi B B, Hotchkiss J A, Krieger S M, et al. (2008) Respiratory sensitization and allergy: current research approaches and needs. Toxicol Appl Pharmacol 226: 1-13). Mechanistically, respiratory allergy is associated with the induction of Th2 cells and increased IgE production by B cells. Crosslinking of FcεR:s by IgE/allergen complexes on granular effector cells, such as mast cells and basophils, leads to the release of proinflammatory molecules (Boverhof et al., 2008, supra; Banks D E, Tarlo S M (2000) Important issues in occupational asthma. Curr Opin Pulm Med 6: 37-42; Sastre J, Vandenplas O, Park H S (2003) Pathogenesis of occupational asthma. Eur Respir J 22: 364-373). The type I hypersensitivity reaction is classically triggered by protein allergens, while low-molecular weight compounds have a propensity to induce Allergic Contact Dermatitis (ACD), a type IV hypersensitivity reaction that has primarily been associated with the induction of Th1 and CD8⁺ effector cells. However, a number of chemicals, such as diisocyanates (Zammit-Tabona M, Sherkin M, Kijek K, Chan H, Chan-Yeung M (1983) Asthma caused by diphenylmethane diisocyanate in foundry workers. Clinical, bronchial provocation, and immunologic studies. Am Rev Respir Dis 128: 226-230), acid anhydrides (Bernstein D I, Patterson R, Zeiss C R (1982) Clinical and immunologic evaluation of trimellitic anhydride-and phthalic anhydride-exposed workers using a questionnaire with comparative analysis of enzyme-linked immunosorbent and radioimmunoassay studies. J Allergy Clin Immunol 69: 311-318), platinum salts (Murdoch R D, Pepys J, Hughes E G (1986) IgE antibody responses to platinum group metals: a large scale refinery survey. Br J Ind Med 43: 37-43), reactive dyes (Docker A, Wattie J M, Topping M D, Luczynska C M, Newman Taylor A J, et al. (1987) Clinical and immunological investigations of respiratory disease in workers using reactive dyes. Br J Ind Med 44: 534-541), and chloramine T (Bourne M S, Flindt M L, Walker J M (1979) Asthma due to industrial use of chloramine. Br Med J 2: 10-12) may induce respiratory sensitization with occupational asthma and rhinitis as a result. Fewer chemicals are known to cause respiratory allergy, compared to those causing contact dermatitis, however, the health impact may still be disastrous as it can be associated with fatal outcomes. While clinical characteristics are similar to those of allergic reactions to proteins, the nature of the responses often remains unanswered.

The REACH (Registration, Evaluation, and Authorisation of Chemicals) regulation requires that all new and existing chemicals within the European Union, involving approximately 30 000 chemicals, should be tested for hazardous effects (Johansson H, Lindstedt M, Albrekt A S, Borrebaeck C A: A genomic biomarker signature can predict skin sensitizers using a cell-based in vitro alternative to animal tests. BMC Genomics 2011, 12:399). As the identification of potential sensitizers currently requires animal testing, the REACH legislation will have a huge impact on the number of animals needed for testing. Further, the 7th Amendment to the Cosmetics Directive (76/768/EEC) posed a ban on animal tests for the majority of cosmetic ingredients for human use, to be in effect by 2009, with the exceptions of some tests by 2013. Thus, development of reliable in vitro alternatives to experimental animals for the assessment of sensitizing capacity of chemicals is urgent.

Methods for risk assessment of chemicals inducing respiratory sensitization are greatly underdeveloped, with no validated assay available to date (Verstraelen S, Bloemen K, Nelissen I, Witters H, Schoeters G, et al. (2008) Cell types involved in allergic asthma and their use in in vitro models to assess respiratory sensitization. Toxicol In Vitro 22: 1419-1431). The main in vivo assay designed for this purpose is the mouse IgE test (Dearman R J, Basketter D A, Kimber I (1992) Variable effects of chemical allergens on serum IgE concentration in mice. Preliminary evaluation of a novel approach to the identification of respiratory sensitizers. J Appl Toxicol 12: 317-323; Dearman R J, Skinner R A, Humphreys N E, Kimber I (2003) Methods for the identification of chemical respiratory allergens in rodents: comparisons of cytokine profiling with induced changes in serum IgE. J Appl Toxicol 23: 199-207). Although showing promising initial results, interlaboratory reproducibility was not sufficient to formally validate this assay, and it is today not routinely used. However, efforts are made to develop cell-based assays for sensitization of the respiratory tract, using both dendritoid cell lines, such as THP-1 (Verstraelen S, Nelissen I, Hooyberghs J, Witters H, Schoeters G, et al. (2009) Gene profiles of THP-1 macrophages after in vitro exposure to respiratory (non-)sensitizing chemicals: identification of discriminating genetic markers and pathway analysis. Toxicol In Vitro 23: 1151-1162.), as well as epithelial cell lines, such as BEAS-2B (Verstraelen S, Nelissen I, Hooyberghs J, Witters H, Schoeters G, et al. (2009) Gene profiles of a human bronchial epithelial cell line after in vitro exposure to respiratory (non-)sensitizing chemicals: identification of discriminating genetic markers and pathway analysis. Toxicology 255: 151-159) and A549 (Verstraelen S, Nelissen I, Hooyberghs J, Witters H, Schoeters G, et al. (2009) Gene profiles of a human alveolar epithelial cell line after in vitro exposure to respiratory (non-)sensitizing chemicals: identification of discriminating genetic markers and pathway analysis. Toxicol Lett 185: 16-22). Furthermore, chemical reactivity assays are being explored within respiratory sensitization, as well as for ACD (Lalko J F, Kimber I, Dearman R J, Gerberick G F, Sarlo K, et al. (2011) Chemical reactivity measurements: potential for characterization of respiratory chemical allergens. Toxicol In Vitro 25: 433-445). However, peptide reactivity has been shown to be a common feature for sensitizers of both skin and respiratory tract, severely complicating discrimination between the two groups.

Dendritic cells (DCs) play key roles in the immune response by bridging the essential connections between innate and adaptive immunity. They can, upon triggering, rapidly produce large amounts of mediators, which influence migration and activation of other cells at the site of inflammation, and selectively respond to various pathogens and environmental factors, by fine-tuning the cellular response through antigen-presentation. Thus, exploring and utilizing the immunological decision-making by DCs during stimulation with sensitizers, could serve as a potent test strategy for prediction of sensitization.

However, multifaceted phenotypes and specialized functions of different DC subpopulations, as well as their wide and scarce distribution, are complicating factors, which impede the employment of primary DCs as a test platform. Hence, there is a real need to establish accurate and reliable in vitro assays that also circumvent the problems associated with variability of and difficulty in obtaining DCs.

### DISCLOSURE OF THE INVENTION

Thus, the development of assays based on the predictability of DC function should preferably rely on alternative cell types or mimics of in vivo DCs. For this purpose, a cell line with DC characteristics would be advantageous, as it constitutes a stable, reproducible and unlimited supply of cells. In terms of DC mimics, differentiated myelomonocytic MUTZ-3 cells are the preferred candidate (Masterson, A. J., C. C. Sombroek, T. D. De Gruijl, Y. M. Graus, H. J. van der Vliet, S. M. Lougheed, A. J. van den Eertwegh, H. M. Pinedo, and R. J. Scheper. 2002. MUTZ-3, a human cell line model for the cytokine-induced differentiation of dendritic cells from CD34+ precursors. Blood 100:701-703.). MUTZ-3 is as an unlimited source of CD34⁺ DC progenitors and it can acquire, upon cytokine stimulation, phenotypes similar to immature DCs or Langerhans-like DCs (Santegoets, S. J., M. W. Schreurs, A. J. Masterson, Y. P. Liu, S. Goletz, H. Baumeister, E. W. Kueter, S. M. Lougheed, A. J. van den Eertwegh, R. J. Scheper, E. Hooijberg, and T. D. de Gruijl. 2006. In vitro priming of tumor-specific cytotoxic T lymphocytes using allogeneic dendritic cells derived from the human MUTZ-3 cell line. Cancer Immunol Immunother 55:1480-1490.), present antigens through CD1d, MHC class I and II and induce specific T-cell proliferation (Masterson, A. J., C. C. Sombroek, T. D. De Gruijl, Y. M. Graus, H. J. van der Vliet, S. M. Lougheed, A. J. van den Eertwegh, H. M. Pinedo, and R. J. Scheper. 2002. MUTZ-3, a human cell line model for the cytokine-induced differentiation of dendritic cells from CD34+ precursors. Blood 100:701-703.). MUTZ-3 also displays a mature transcriptional and phenotypic profile upon stimulation with inflammatory mediators (Larsson K, Lindstedt M, and Borrebaeck C A K. Functional and transcriptional profiling of MUTZ-3. A myeloid cell line acting as a model for dendritic cells. Immunology. 2006 Feb; 117(2):156-66.)

The present inventors have developed a novel test principle for prediction of respiratory sensitizers. It has surprisingly been found that respiratory sensitizers can be accurately identified/predicted using DC progenitor cells, such as MUTZ-3 cells, without further differentiation in a process whereby the cells are stimulated with a panel of sensitizing chemicals, non-sensitizing chemicals, and/or other controls (e.g. vehicle controls comprising diluent only, such as DMSO and/or distilled water). This was found to substantially simplify and improve the reproducibility of the procedure.

Hence, a first aspect of the present invention provides a method for identifying agents capable of inducing respiratory sensitization in a mammal comprising or consisting of the steps of:
a) exposing a population of dendritic cells or a population of dendritic-like cells to a test agent; and
b) measuring in the cells the expression of one or more biomarker(s) selected from the group defined in Table 1,

wherein the expression in the cells of the one or more biomarkers measured in step (b) is indicative of the respiratory sensitizing effect of the test agent.

By “agents capable of inducing respiratory sensitization” we mean any agent capable of inducing and triggering a Type I immediate hypersensitivity reaction in the respiratory tract of a mammal. Preferably the mammal is a human. Preferably, the Type I immediate hypersensitivity reaction is DC-mediated and/or involves the differentiation of T cells into Th2 cells. Preferably the Type I immediate hypersensitivity reaction results in humoral immunity and/or respiratory allergy.

The conducting zone of the mammalian lung contains the trachea, the bronchi, the bronchioles, and the terminal bronchioles. The respiratory zone contains the respiratory bronchioles, the alveolar ducts, and the alveoli. The conducting zone is made up of airways, has no gas exchange with the blood, and is reinforced with cartilage in order to hold open the airways. The conducting zone humidifies inhaled air and warms it to 37° C. (99° F.). It also cleanses the air by removing particles via cilia located on the walls of all the passageways. The respiratory zone is the site of gas exchange with blood.

In one embodiment, the “agents capable of inducing sensitization of mammalian skin” is an agent capable of inducing and triggering a Type I immediate hypersensitivity reaction at a site of lung epithelium in a mammal. Preferably, the site of lung epithelium is in the respiratory zone of the lung, but may alternatively or additionally be in the conductive zone of the lung.

The mammal may be any domestic or farm animal. Preferably, the mammal is a rat, mouse, guinea pig, cat, dog, horse or a primate. Most preferably, the mammal is human.

Dendritic cells (DCs) are immune cells forming part of the mammalian immune system. Their main function is to process antigen material and present it on the surface to other cells of the immune system (i.e., they function as antigen-presenting cells), bridging the innate and adaptive immune systems.

Dendritic cells are present in tissues in contact with the external environment, such as the skin (where there is a specialized dendritic cell type called Langerhans cells) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections, the dendrites. While similar in appearance, these are distinct structures from the dendrites of neurons. Immature dendritic cells are also called veiled cells, as they possess large cytoplasmic ‘veils’ rather than dendrites.

By “dendritic-like cells” we mean non-dendritic cells that exhibit functional and phenotypic characteristics specific to dendritic cells such as morphological characteristics, expression of costimulatory molecules and MHC class II molecules, and the ability to pinocytose macromolecules and to activate resting T cells.

In one embodiment, the dendritic-like cells are CD34⁺ dendritic cell progenitors. Optionally, the CD34⁺ dendritic cell progenitors can acquire, upon cytokine stimulation, the phenotypes of presenting antigens through CD1d, MHC class I and II, induce specific T-cell proliferation, and/or displaying a mature transcriptional and phenotypic profile upon stimulation with inflammatory mediators (i.e. similar phenotypes to immature dendritic cells or Langerhans-like dendritic cells).

Dendritic cells may be recognized by function, by phenotype and/or by gene expression pattern, particularly by cell surface phenotype. These cells are characterized by their distinctive morphology, high levels of surface MHC-class II expression and ability to present antigen to CD4+ and/or CD8+ T cells, particularly to naïve T cells (Steinman et al. (1991) Ann. Rev. Immunol. 9: 271).

The cell surface of dendritic cells is unusual, with characteristic veil-like projections, and is characterized by expression of the cell surface markers CD11c and MHC class II. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3Ra), CD134, CD137, CD150, CD153, CD162, CXCR1, CXCR2, CXCR4, DCIR, DC-LAMP, DC-SIGN, DEC205, E-cadherin, Langerin, Mannose receptor, MARCO, TLR2, TLR3 TLR4, TLR5, TLR6, TLR9, and several lectins.

The patterns of expression of these cell surface markers may vary along with the maturity of the dendritic cells, their tissue of origin, and/or their species of origin. Immature dendritic cells express low levels of MHC class II, but are capable of endocytosing antigenic proteins and processing them for presentation in a complex with MHC class II molecules. Activated dendritic cells express high levels of MHC class 11, ICAM-1 and CD86, and are capable of stimulating the proliferation of naive allogeneic T cells, e. g. in a mixed leukocyte reaction (MLR).

Functionally, dendritic cells or dendritic-like cells may be identified by any convenient assay for determination of antigen presentation. Such assays may include testing the ability to stimulate antigen- primed and/or naive T cells by presentation of a test antigen, followed by determination of T cell proliferation, release of IL-2, and the like.

By “expression” we mean the level or amount of a gene product such as mRNA or protein.

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR), nanoarray, microarray, macroarray, autoradiography and in situ hybridisation.

In one embodiment the method further comprises the steps of:
c) exposing a separate population of the dendritic cells or dendritic-like cells to one or more negative control agent that is not a respiratory sensitizer in mammals; and
d) measuring in the cells the expression of the one or more biomarker(s) measured in step (b)
wherein the test agent is identified as a respiratory sensitizer in the event that the presence and/or amount in the test sample of the one or more biomarker measured in step (b) is different from the presence and/or amount in the control sample of the one or more biomarkers measured in step (d).

By “is different from the presence and/or amount in the control sample of the one or more proteins measured in step (b)” we mean that the presence and/or amount in the test sample differs from that of the one or more negative control sample in a statistically significant manner. Preferably the expression of the one or more biomarker in the cell population exposed to the test agent is:
less than or equal to 80% of that of the cell population exposed to the negative control agent, for example, no more than 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or 0% of that of the cell population exposed to the negative control agent; or
at least 120% of that of the cell population exposed to the negative control agent, for example, at least 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 225%, 250%, 275%, 300%, 325%, 350%, 375%, 400%, 425%, 450%, 475% or at least 500% of that of the cell population exposed to the negative control agent

The one or more negative control agent may comprise or consist of one or more agent selected from the group consisting of 1-butanol, 4-aminobenzoic acid, chlorobenzene, dimethyl formamide, ethyl vanillin, isopropanol, methyl salicylate, propylene glycol, potassium permanganate, Tween 80™ (polyoxyethylene (20) sorbitan monooleate) and zinc sulphate (i.e., the group of non-sensitizers defined in Table 2).

The negative control agent may be a solvent for use with the test or control agents of the invention. Hence, the negative control may be DMSO and/or distilled water.

Alternatively or additionally, the expression of the one or more biomarkers measured in step (b) of the dendritic cells or dendritic-like cells prior to test agent exposure is used as a negative control.

A further embodiment comprises the steps of:
e) exposing a separate population of the dendritic cells or dendritic-like cells to one or more positive control agent that is a respiratory sensitizer in a mammal; and
f) measuring in the cells the expression of the one or more biomarker(s) measured in step (b)

wherein the test agent is identified as a respiratory sensitizer in the event that the presence and/or amount in the test sample of the one or more biomarker measured in step (f) corresponds to the presence and/or amount in the one or more positive control sample of the one or more biomarker measured in step (b).

By “corresponds to the expression in the one or more positive control sample” we mean the expression of the one or more biomarker in the cell population exposed to the test agent is identical to, or does not differ significantly from, that of the cell population exposed to the one more positive control agent. Preferably the expression of the one or more biomarker in the cell population exposed to the test agent is between 81% and 119% of that of the cell population exposed to the one more positive control agent, for example, greater than or equal to 82%, 83%, 84%, 85%, 86%, 87%, 88%, 89%, 90%, 91%, 92%, 93%, 94%, 95%, 96%, 97%, 98% or 99% of that of the cell population exposed to the one more positive control agent, and less than or equal to 101%, 102%, 103%, 104%, 105%, 106%, 107%, 108%, 109%, 110%, 111%, 112%, 113%, 114%, 115%, 116%, 117%, 118% or 119% of that of the cell population exposed to the one more positive control agent.

Hence, the method according to the first aspect of the invention may include measuring OR5B21 expression. The method may include measuring SLC7A7 expression.

The method may include measuring PIP3-E expression. The method may include measuring BTNL8 expression. The method may include measuring CLEC4A expression. The method may include measuring HIST4H4 expression. The method may include measuring YKT6 expression. The method may include measuring FLJ32679///GOLGA8G///GOLGA8E expression. The method may include measuring PACSIN3 expression. The method may include measuring PDE1B expression. The method may include measuring NQO1 expression. The method may include measuring CAMK1D expression. The method may include measuring MYB expression. The method may include measuring ENST00000387396 expression. The method may include measuring GRK5 expression.

The method may include measuring CD86 expression. The method may include measuring CD1A expression. The method may include measuring WWOX expression. The method may include measuring IKZF2 expression. The method may include measuring FUCA1 expression. The method may include measuring C10orf76 expression. The method may include measuring AMICA1 expression. The method may include measuring PDPK2///PDPK1 expression. The method may include measuring AZU1 expression. The method may include measuring ACN9 expression. The method may include measuring PDPN expression. The method may include measuring LOC642587 expression. The method may include measuring SEC61A2 expression. The method may include measuring ELA2 expression. The method may include measuring BMP2K expression. The method may include measuring HCCS expression. The method may include measuring CXorf26 expression. The method may include measuring TYSND1 expression. The method may include measuring CARS expression. The method may include measuring NECAP1 expression. The method may include measuring CDH26 expression. The method may include measuring SERPINB1 expression. The method may include measuring STEAP4 expression. The method may include measuring TXNIP expression. The method may include measuring ENST00000386628 expression. The method may include measuring C12orf35 expression. The method may include measuring HMGA2 expression. The method may include measuring KRT16 expression. The method may include measuring GGTLC2 expression. The method may include measuring ENST00000386437 expression. The method may include measuring OSBPL11 expression. The method may include measuring FAM71F1 expression. The method may include measuring ATP6V1B2 expression. The method may include measuring LOC128102 expression. The method may include measuring TBX19 expression. The method may include measuring NID1 expression. The method may include measuring LPXN expression. The method may include measuring C15orf45 expression. The method may include measuring RNF111 expression. The method may include measuring ENST00000386861 expression. The method may include measuring CD33 expression. The method may include measuring TANK expression. The method may include measuring ANKRD44 expression. The method may include measuring WDFY1 expression. The method may include measuring SDC4 expression. The method may include measuring TMPRSS11B expression. The method may include measuring AFF4 expression. The method may include measuring HBEGF expression. The method may include measuring XK expression. The method may include measuring SLAMF7 expression. The method may include measuring S100A4 expression. The method may include measuring MPZL3 expression. The method may include measuring GENSCAN00000044853 expression. The method may include measuring TRAV8-3 expression. The method may include measuring LOC100131497 expression. The method may include measuring KIAA1468 expression. The method may include measuring SPHK2 expression. The method may include measuring ENST00000309260 expression. The method may include measuring CCR6 expression. The method may include measuring GSTA3 expression. The method may include measuring RALA expression. The method may include measuring C7orf53 expression. The method may include measuring AF480566 expression. The method may include measuring CERCAM expression. The method may include measuring hsa-mir-147 expression. The method may include measuring NFYC expression. The method may include measuring CD53 expression. The method may include measuring PSEN2 expression. The method may include measuring CISD1 expression. The method may include measuring SCD expression. The method may include measuring MED19 expression. The method may include measuring SYT17 expression. The method may include measuring KRT16///LOC400578///MGC102966 expression. The method may include measuring C18orf51 expression. The method may include measuring CD79A expression. The method may include measuring C19orf56 expression. The method may include measuring AGFG1 expression. The method may include measuring FOXP1 expression. The method may include measuring TLR6 expression. The method may include measuring SUSD3 expression. The method may include measuring ENST00000387842 expression. The method may include measuring ENST00000387842 expression. The method may include measuring GPA33 expression. The method may include measuring CDC123 expression. The method may include measuring C10orf11 expression. The method may include measuring ENST00000322493 expression. The method may include measuring PTMAP7 expression. The method may include measuring ARRDC4 expression. The method may include measuring ENST00000388199 expression. The method may include measuring ENST00000388437 expression. The method may include measuring KRT9 expression. The method may include measuring ENST00000379371 expression. The method may include measuring HDAC4 expression. The method may include measuring CD200 expression. The method may include measuring PAPSS1 expression. The method may include measuring ORAI2 expression. The method may include measuring AK124536 expression. The method may include measuring ZBTB10 expression. The method may include measuring ENST00000387422 expression. The method may include measuring RAB9A expression. The method may include measuring 7895613 expression. The method may include measuring DRD5 expression. The method may include measuring CNR2 expression. The method may include measuring OIT3 expression. The method may include measuring ENST00000386981 expression. The method may include measuring C10orf90 expression. The method may include measuring OR52D1 expression. The method may include measuring ZNF214 expression. The method may include measuring ENST00000386959 expression. The method may include measuring ART4 expression. The method may include measuring RCBTB2 expression. The method may include measuring HOMER2 expression. The method may include measuring WWP2 expression. The method may include measuring WDR24 expression. The method may include measuring MED31 expression. The method may include measuring CALM2 expression. The method may include measuring DLX2 expression. The method may include measuring BTBD3 expression. The method may include measuring ENST00000339367 expression. The method may include measuring TBCA expression. The method may include measuring GIN1 expression. The method may include measuring NOL7 expression. The method may include measuring ENST00000402365 expression. The method may include measuring C7orf28B///C7orf28A expression. The method may include measuring DPP7 expression. The method may include measuring hCG_1749005 expression. The method may include measuring PNPLA4 expression. The method may include measuring USP51 expression. The method may include measuring HLA-DQA1///HLA-DRA expression. The method may include measuring FAAH expression. The method may include measuring GDAP2 expression. The method may include measuring CD48 expression. The method may include measuring PTPRJ expression. The method may include measuring EXPH5 expression. The method may include measuring RPS26///LOC728937///RPS26L///hCG_2033311 expression. The method may include measuring ALDH2 expression. The method may include measuring CALM1 expression. The method may include measuring NOX5///SPESP1 expression. The method may include measuring RHBDL1 expression. The method may include measuring CYLD expression. The method may include measuring OSBPL1A expression. The method may include measuring GYPC expression. The method may include measuring RQCD1 expression. The method may include measuring RBM44 expression. The method may include measuring ENST00000384680 expression. The method may include measuring C3orf58 expression. The method may include measuring MFSD1 expression. The method may include measuring HACL1 expression. The method may include measuring SATB1 expression. The method may include measuring USP4 expression. The method may include measuring ENST00000410125 expression. The method may include measuring ENST00000384055 expression. The method may include measuring L7R expression. The method may include measuring ENST00000364497 expression. The method may include measuring FAM135A expression. The method may include measuring CD164 expression. The method may include measuring DYNLT1 expression. The method may include measuring NRCAM expression. The method may include measuring ZNF596 expression. The method may include measuring ENST00000332418 expression. The method may include measuring TCEAL3///TCEAL6 expression. The method may include measuring SNAPIN expression. The method may include measuring DENND2D expression. The method may include measuring SAMD8 expression. The method may include measuring LHPP expression. The method may include measuring SLC37A2 expression. The method may include measuring FLI1///EWSR1 expression. The method may include measuring OR9G4 expression. The method may include measuring LOC338799 expression. The method may include measuring HEXDC expression. The method may include measuring NOTUM expression. The method may include measuring MCOLN1 expression. The method may include measuring PRKACA expression. The method may include measuring CRIM1 expression. The method may include measuring CECR5 expression. The method may include measuring RNF13 expression. The method may include measuring 40969 expression. The method may include measuring ZNF366 expression. The method may include measuring ENST00000410754 expression. The method may include measuring GIMAP5 expression. The method may include measuring ENST00000362484 expression. The method may include measuring TFE3 expression. The method may include measuring RHOU expression. The method may include measuring MED8 expression. The method may include measuring CASQ2 expression. The method may include measuring NUDT5 expression. The method may include measuring C11orf73 expression. The method may include measuring PAK1 expression. The method may include measuring PRSS21 expression. The method may include measuring ENST00000332418 expression. The method may include measuring BTBD12 expression. The method may include measuring DHRS13 expression. The method may include measuring CCDC102B expression. The method may include measuring BCL2 expression. The method may include measuring ZNF211///ZNF134 expression. The method may include measuring NDUFV2 expression. The method may include measuring MYCN expression. The method may include measuring ENST00000385528 expression. The method may include measuring ENST00000264275 expression. The method may include measuring CASP8 expression. The method may include measuring RTN4 expression. The method may include measuring PLCG1 expression. The method may include measuring MGC42105 expression. The method may include measuring EMB expression. The method may include measuring ENST00000386433 expression. The method may include measuring COL21A1 expression. The method may include measuring LRP12 expression. The method may include measuring LMNA expression. The method may include measuring ENST00000385567 expression. The method may include measuring ENST00000362863 expression. The method may include measuring ZNF503 expression. The method may include measuring NLRX1 expression. The method may include measuring ENST00000391173 expression. The method may include measuring NDRG2 expression. The method may include measuring TRAF7 expression. The method may include measuring KRT40 expression. The method may include measuring KRT40 expression. The method may include measuring DRD5 expression. The method may include measuring ZC3H8 expression. The method may include measuring MMP9 expression. The method may include measuring PLTP expression. The method may include measuring ENST00000362686 expression. The method may include measuring SPEF2 expression. The method may include measuring LRRC16A expression. The method may include measuring FBXO9 expression. The method may include measuring EEPD1 expression. The method may include measuring FCN1 expression. The method may include measuring EFNA3 expression. The method may include measuring ENST00000314893 expression. The method may include measuring TMEM19 expression. The method may include measuring PLXNC1 expression. The method may include measuring NHLRC3 expression. The method may include measuring MBNL2 expression. The method may include measuring EIF5 expression. The method may include measuring PLEKHG4 expression. The method may include measuring COPS3 expression. The method may include measuring FAM171A2 expression. The method may include measuring LOC653653///AP1S2 expression. The method may include measuring VAPA expression. The method may include measuring MATK expression. The method may include measuring ACTR2 expression. The method may include measuring BPI expression. The method may include measuring ERG expression. The method may include measuring LAMB2 expression. The method may include measuring BC090058 expression. The method may include measuring PHTF2 expression. The method may include measuring ENST00000333261 expression. The method may include measuring C8orf55 expression. The method may include measuring PDE7A expression. The method may include measuring NAPRT1 expression. The method may include measuring HLA-DRA expression. The method may include measuring SLC22A15 expression. The method may include measuring FCGR1A///FCGR1B///FCGR1C expression. The method may include measuring SLC27A3 expression. The method may include measuring ID3 expression. The method may include measuring TBCEL expression. The method may include measuring FAM138D expression. The method may include measuring POMP expression. The method may include measuring SNN expression. The method may include measuring MED13 expression. The method may include measuring ZFP36L2 expression. The method may include measuring UXS1 expression. The method may include measuring CD40 expression. The method may include measuring ENST00000362620 expression. The method may include measuring GGT5 expression. The method may include measuring BC035666 expression. The method may include measuring G6PD expression. The method may include measuring ENST00000384272 expression. The method may include measuring CLCC1 expression. The method may include measuring SCGB2A1 expression. The method may include measuring GAA expression. The method may include measuring SERPINB2 expression. The method may include measuring GPI expression. The method may include measuring LASS6 expression. The method may include measuring EIF4A2 expression. The method may include measuring HLA-DRA expression. The method may include measuring ENST00000385586 expression. The method may include measuring ANXA2P2 expression. The method may include measuring FANCG expression. The method may include measuring FAM53B expression. The method may include measuring RFXAP expression. The method may include measuring UBR1 expression. The method may include measuring TBC1D2B expression. The method may include measuring SERPINB10 expression. The method may include measuring SEC23B expression. The method may include measuring MN1 expression. The method may include measuring CRTAP expression.

The method may comprise or consist of measuring, in step (b), the expression of one or more biomarkers defined in Table 1A, for example, at least 2 of the biomarkers defined in Table 1A. Hence, the method may comprise measuring the expression of OR5B21. The method may comprise measuring the expression of SLC7A7. In a preferred embodiment, the method comprises or consists of measuring the expression of OR5B21 and SLC7A7 in step (b).

The method may additionally or alternatively comprise or consist of, measuring in step (b) the expression of one or more biomarkers defined in Table 1B, for example, 2, 3, 4, 5, 6, 7, 8, 9, 10, 11, 12 or 13 of the biomarkers defined in Table 1B.

The method may additionally or alternatively comprise or consist of, measuring in step (b) the expression of one or more biomarkers defined in Table 10, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286 or 287 of the biomarkers defined in Table 10.

Thus, the expression of all of the biomarkers defined in Table 1A and/or all of the biomarkers defined in Table 1B and/or all of the biomarkers defined in Table 10 may be measured in step (b). Hence, the method may comprise or consist of measuring in step (b) all of the biomarkers defined in Table 1.

In a preferred embodiment, step (b) comprises or consists of measuring the expression of a nucleic acid molecule encoding the one or more biomarker(s). The nucleic acid molecule may be a cDNA molecule or an mRNA molecule. Preferably, the nucleic acid molecule is an mRNA molecule. However, the nucleic acid molecule may be a cDNA molecule.

In one embodiment the expression of the one or more biomarker(s) in step (b) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably, the expression of the one or more biomarker(s) is measured using a DNA microarray.

The method may comprise measuring the expression of the one or more biomarker(s) in step (b) using one or more binding moieties, each capable of binding selectively to a nucleic acid molecule encoding one of the biomarkers identified in Table 1. In one embodiment the one or more binding moieties each comprise or consist of a nucleic acid molecule. In a further embodiment the one or more binding moieties each comprise or consist of DNA, RNA, PNA, LNA, GNA, TNA or PMO. Preferably, the one or more binding moieties each comprise or consist of DNA. In one embodiment, the one or more binding moieties are 5 to 100 nucleotides in length. However, in an alternative embodiment, they are 15 to 35 nucleotides in length.

Suitable binding agents (also referred to as binding molecules) may be selected or screened from a library based on their ability to bind a given nucleic acid, protein or amino acid motif, as discussed below.

In a preferred embodiment, the binding moiety comprises a detectable moiety.

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Hence, the detectable moiety may be selected from the group consisting of: a fluorescent moiety; a luminescent moiety; a chemiluminescent moiety; a radioactive moiety (for example, a radioactive atom); or an enzymatic moiety. Preferably, the detectable moiety comprises or consists of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In an alternative preferred embodiment, the detectable moiety of the binding moiety is a fluorescent moiety.

The radio- or other labels may be incorporated into the biomarkers present in the samples of the methods of the invention and/or the binding moieties of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that biomarkers in the sample(s) to be tested may be labelled with a moiety which indirectly assists with determining the presence, amount and/or location of said proteins. Thus, the moiety may constitute one component of a multicomponent detectable moiety. For example, the biomarkers in the sample(s) to be tested may be labelled with biotin, which allows their subsequent detection using streptavidin fused or otherwise joined to a detectable label.

The method provided in the first aspect of the present invention may comprise or consist of, in step (b), determining the expression of the protein of the one or more biomarker defined in Table 1. The method may comprise measuring the expression of the one or more biomarker(s) in step (b) using one or more binding moieties each capable of binding selectively to one of the biomarkers identified in Table 1. The one or more binding moieties may comprise or consist of an antibody or an antigen-binding fragment thereof such as a monoclonal antibody or fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art.

We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5):949-56).

In addition, display systems have been developed utilising linkage of the polypeptide product to its encoding mRNA in so-called ribosome display systems (Hanes & Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The antibody or antigen-binding fragment may be selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Preferably, the antibody or antigen-binding fragment is a single chain Fv (scFv).

The one or more binding moieties may alternatively comprise or consist of an antibody-like binding agent, for example an affibody or aptamer.

By “scFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

The one or more protein-binding moieties may comprise a detectable moiety. The detectable moiety may be selected from the group consisting of a fluorescent moiety, a luminescent moiety, a chemiluminescent moiety, a radioactive moiety and an enzymatic moiety.

In a further embodiment of the methods of the invention, step (b) may be performed using an assay comprising a second binding agent capable of binding to the one or more proteins, the second binding agent also comprising a detectable moiety. Suitable second binding agents are described in detail above in relation to the first binding agents.

Thus, the proteins of interest in the sample to be tested may first be isolated and/or immobilised using the first binding agent, after which the presence and/or relative amount of said biomarkers may be determined using a second binding agent.

In one embodiment, the second binding agent is an antibody or antigen-binding fragment thereof; typically a recombinant antibody or fragment thereof. Conveniently, the antibody or fragment thereof is selected from the group consisting of: scFv; Fab; a binding domain of an immunoglobulin molecule. Suitable antibodies and fragments, and methods for making the same, are described in detail above.

Alternatively, the second binding agent may be an antibody-like binding agent, such as an affibody or aptamer.

Alternatively, where the detectable moiety on the protein in the sample to be tested comprises or consists of a member of a specific binding pair (e.g. biotin), the second binding agent may comprise or consist of the complimentary member of the specific binding pair (e.g. streptavidin).

Where a detection assay is used, it is preferred that the detectable moiety is selected from the group consisting of: a fluorescent moiety; a luminescent moiety; a chemiluminescent moiety; a radioactive moiety; an enzymatic moiety. Examples of suitable detectable moieties for use in the methods of the invention are described above.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Thus, in one embodiment the assay is an ELISA (Enzyme Linked Immunosorbent Assay) which typically involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an alternative embodiment, the assay used for protein detection is conveniently a fluorometric assay. Thus, the detectable moiety of the second binding agent may be a fluorescent moiety, such as an Alexa fluorophore (for example Alexa-647).

Preferably, step (b) is performed using an array. The array may be a bead-based array or a surface-based array. The array may be selected from the group consisting of: macroarray; microarray; nanoarray.

In on embodiment, the method is for identifying agents capable of inducing a respiratory hypersensitivity response. Preferably, the hypersensitivity response is a humoral hypersensitivity response, for example, a type I hypersensitivity response. Preferably, the method is for identifying agents capable of inducing respiratory allergy.

In one embodiment, the population of dendritic cells or population of dendritic-like cells is a population of dendritic cells. Preferably, the dendritic cells are primary dendritic cells. Preferably, the dendritic cells are myeloid dendritic cells.

The population of dendritic cells or dendritic-like cells is preferably mammalian in origin. Preferably, the mammal is a rat, mouse, guinea pig, cat, dog, horse or a primate. Most preferably, the mammal is human.

In an embodiment the population of dendritic cells or population of dendritic-like cells is a population of dendritic-like cells, preferably myeloid dendritic-like cells.

In one embodiment, the dendritic-like cells express at least one of the markers selected from the group consisting of CD54, CD86, CD80, HLA-DR, CD14, CD34 and CD1a, for example, 2, 3, 4, 5, 6 or 7 of the markers. In a further embodiment, the dendritic-like cells express the markers CD54, CD86, CD80, HLA-DR, CD14, CD34 and CD1a.

In a further embodiment, the dendritic-like cells may be derived from myeloid dendritic cells. Preferably the dendritic-like cells are myeloid leukaemia-derived cells. Preferably, the myeloid leukaemia-derived cells are selected from the group consisting of KG-1, THP-1, U-937, HL-60, Monomac-6, AML-193 and MUTZ-3. Most preferably, dendritic-like cells are MUTZ-3 cells. MUTZ-3 cells are human acute myelomonocytic leukemia cells that were available from 15 May 1995 under deposit number ACC 295 from Deutsche Sammlung für Mikroorganismen and Zellkulturen GmbH (DSMZ), Inhoffenstraβe 7B, Braunschweig, Germany (www.dsmz.de).

In one embodiment, the dendritic-like cells, after stimulation with cytokine, present antigens through CD1d, MHC class I and II and/or induce specific T-cell proliferation.

In one embodiment, the one or more negative control agent comprises or consists of one or more agent selected from the group consisting of 1-butanol, 4-aminobenzoic acid, chlorobenzene, dimethyl formamide, ethyl vanillin, isopropanol, methyl salicylate, propylene glycol, potassium permanganate, Tween 80™ (polyoxyethylene (20) sorbitan monooleate) and zinc sulphate (i.e., the group of non-sensitizers defined in Table 2). Hence, step (c) may comprise or consist of exposing separate populations of the dendritic cells or dendritic-like cells to each of the negative control agents defined in Table 2.

The method may comprise or consist of the use of at least 2 negative control agents (i.e. non-sensitizing agents), for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 negative control agents.

In another embodiment, the one or more positive control agent comprises or consists of one or more agent selected from the group consisting of ammonium hexachloroplatinate, ammonium persulfate, glutaraldehyde, hexamethylen diisocyanate, maleic anhydride, methylene diphenol diisocyanate, phtalic anhydride, toluendiisocyanate and trimellitic anhydride (i.e., the group of sensitizers defined in Table 2). Hence, step (d) may comprise or consist of exposing separate populations of the dendritic cells or dendritic-like cells to each of the positive control agents defined in Table 2.

The method may comprise or consist of the use of at least 2 positive control (i.e. sensitizing agents), for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 positive control agents.

Hence, in one embodiment, the method is indicative of whether the test agent is or is not a respiratory sensitizing agent. In alternative or additional embodiment, the method is indicative of the respiratory sensitizing potency of the sample to be tested.

Thus, in one embodiment, the method is indicative of the sensitizer potency of the test agent (i.e., that the test agent is either, a non-sensitizer, a weak sensitizer, a moderate sensitizer, a strong sensitizer or an extreme sensitizer). The decision value and distance in PCA correlates with sensitizer potency.

Alternatively or additionally, test agent potency may be determined by, in step (e), providing:
(i) one or more extreme respiratory sensitizer positive control agent;
(ii) one or more strong respiratory sensitizer positive control agent;
(iii) one or more moderate respiratory sensitizer positive control agent; and/or
(iv) one or more weak respiratory sensitizer positive control agent,

wherein the test agent is identified as an extreme respiratory sensitizer in the event that the presence and/or amount in the test sample of the one or more biomarker measured in step (b) corresponds to the presence and/or amount in the extreme positive control sample (where present) of the one or more biomarker measured in step (f); and/or is different from the presence and/or amount in the strong, moderate, weak and/or negative control sample (where present) of the one or more biomarkers measured in step (f),

wherein the test agent is identified as a strong respiratory sensitizer in the event that the presence and/or amount in the test sample of the one or more biomarker measured in step (b) corresponds to the presence and/or amount in the strong positive control sample (where present) of the one or more biomarker measured in step (f); and/or is different from the presence and/or amount in the extreme, moderate, weak and/or negative control sample (where present) of the one or more biomarkers measured in step (f),

wherein the test agent is identified as a moderate respiratory sensitizer in the event that the presence and/or amount in the test sample of the one or more biomarker measured in step (b) corresponds to the presence and/or amount in the moderate positive control sample (where present) of the one or more biomarker measured in step (f); and/or is different from the presence and/or amount in the extreme, strong, weak and/or negative control sample (where present) of the one or more biomarkers measured in step (f), and

wherein the test agent is identified as a weak respiratory sensitizer in the event that the presence and/or amount in the test sample of the one or more biomarker measured in step (b) corresponds to the presence and/or amount in the weak positive control sample (where present) of the one or more biomarker measured in step (f); and/or is different from the presence and/or amount in the extreme, strong, moderate and/or negative control sample (where present) of the one or more biomarkers measured in step (f).

Hence, step (e) may comprise or consist of providing the following categories of respiratory sensitizer positive control:
(a) extreme, strong, moderate and weak;
(b) strong, moderate and weak;
(c) extreme, moderate and weak;
(d) extreme, strong and moderate;
(e) extreme and strong;
(f) strong and moderate;
(g) moderate and weak;
(h) strong and weak;
(i) extreme and moderate;
(j) extreme and weak;
(k) extreme;
(l) strong;
(m) moderate;
(n) weak.

Negative and positive controls may be classified as respiratory non-sensitizers or respiratory sensitizers, respectively, based on clinical observations in humans.

Alternatively or additionally the method may comprise comparing the expression of the one or more biomaker measured in step (b) with one or more predetermined reference value representing the expression of the one or more biomarker measured in step (c) and/or step (e).

Generally, respiratory sensitizing agents are determined with an ROC AUC of at least 0.55, for example with an ROC AUC of at least, 0.60, 0.65, 0.70, 0.75, 0.80, 0.85, 0.90, 0.95, 0.96, 0.97, 0.98, 0.99 or with an ROC AUC of 1.00. Preferably, skin sensitizing agents are determined with an ROC AUC of at least 0.85, and most preferably with an ROC AUC of 1.

Typically, agents capable of inducing respiratory sensitization are identified using a support vector machine (SVM), such as those available from http://cran.r-project.org/web/packages/e1071/index.html (e.g. e1071 1.5-24). However, any other suitable means may also be used. SVMs may also be used to determine the ROC AUCs of biomarker signatures comprising or consisting of one or more Table 1 biomarkers as defined herein.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles of known agents (namely, known sensitizing or non-sensitizing agents). By running such training samples, the SVM is able to learn what biomarker profiles are associated with agents capable of inducing sensitization. Once the training process is complete, the SVM is then able whether or not the biomarker sample tested is from a sensitizing agent or a non-sensitizing agent.

This allows test agents to be classified as sensitizing or non-sensitizing. Moreover, by training the SVM with sensitizing agents of known potency (i.e. non-sensitizing, weak, moderate, strong or extreme sensitizing agents), the potency of test agents can also be identified comparatively.

However, this training procedure can be by-passed by pre-programming the SVM with the necessary training parameters. For example, agents capable of inducing sensitization can be identified according to the known SVM parameters using the SVM algorithm detailed in Table 3, based on the measurement of all the biomarkers listed in Table 1.

It will be appreciated by skilled persons that suitable SVM parameters can be determined for any combination of the biomarkers listed Table 1 by training an SVM machine with the appropriate selection of data (i.e. biomarker measurements from cells exposed to known sensitizing and/or non-sensitizing agents). Alternatively, the Table 1 biomarkers may be used to identify agents capable of inducing respiratory sensitization according to any other suitable statistical method known in the art.

Alternatively, the Table 1 data may be used to identify agents capable of inducing respiratory sensitization according to any other suitable statistical method known in the art (e.g., ANOVA, ANCOVA, MANOVA, MANCOVA, Multivariate regression analysis, Principal components analysis (PCA), Factor analysis, Canonical correlation analysis, Canonical correlation analysis, Redundancy analysis Correspondence analysis (CA; reciprocal averaging), Multidimensional scaling, Discriminant analysis, Linear discriminant analysis (LDA), Clustering systems, Recursive partitioning and Artificial neural networks).

Preferably, the method of the invention has an accuracy of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive chemicals that are correctly classified as positives, and by “specificity” we mean the proportion of all negative chemicals that are correctly classified as negatives.

In one embodiment, the method of the first aspect of the invention comprises concurrently or consecutively performing a method for identifying agents capable of inducing sensitization of mammalian skin described in PCT publication number WO 2012/056236 which is incorporated herein by reference. Preferably the method for identifying agents capable of inducing sensitization of mammalian skin is performed concurrently with the method of the first aspect of the present invention (i.e., determining whether a test compound is a skin and/or respiratory sensitizer by measuring relevant marker expression in the same cell sample(s) exposed to the test agent).

A second aspect of the invention provides an array for use in the method of the first aspect of the invention (or any embodiment or combination of embodiments thereof), the array comprising one or more binding moieties as defined above. In one embodiment, the binding moieties are (collectively) capable of binding to all of the biomarkers defined in Table 1A. In a further embodiment, the binding moieties are (collectively) capable of binding to all of the biomarkers defined in Table 3B. In a still further embodiment, the binding moieties are (collectively) capable of binding to all of the biomarkers defined in Table 3B. Preferably, the binding moieties are (collectively) capable of binding to all of the biomarkers defined in Table 1.

The binding moieties may be immobilised.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μm, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology; see Examples below.

A third aspect of the present invention provides the use of one or more (preferably two or more) biomarkers selected from the group defined in Table 1A Table 1B and/or Table 1C in combination for identifying hypersensitivity response sensitising agents. Preferably, all of the biomarkers defined in Table 1A and Table 1B are used collectively for identifying hypersensitivity response sensitising agents. Preferably, the use is consistent with the method described in the first aspect of the invention, and the embodiments described therein.

A fourth aspect of the invention provides an analytical kit for use in a method according the first aspect of the invention, comprising or consisting of:
A) an array according to the second aspect of the invention; and
B) instructions for performing the method according to the first aspect of the invention (optional).

The analytical kit may comprise one or more control agents. Preferably, the analytical kit comprises or consists of the above features, together with one or more negative control agents and/or one or more positive control agents.

A fifth aspect of the invention provides a method of treating or preventing a respiratory type I hypersensitivity reaction (such as respiratory asthma) in a patient comprising the steps of:
(a) providing one or more test agent that the patient is or has been exposed to;
(b) determining whether the one or more test agent provided in step (a) is a respiratory sensitizer using a method provided in the first aspect of the present invention; and
(c) where one or more test agent is identified as a respiratory sensitizer, reducing or preventing exposure of the patient to the one or more test agent identified as a respiratory sensitizer.

Preferably, the one or more test agent that the patient is or has been exposed to is an agent that the patient is presently exposed to at least once a month, for example, at least once every two weeks, at least once every week, or at least once every day.

Preferred, non-limiting examples which embody certain aspects of the invention will now be described, with reference to the following figures:

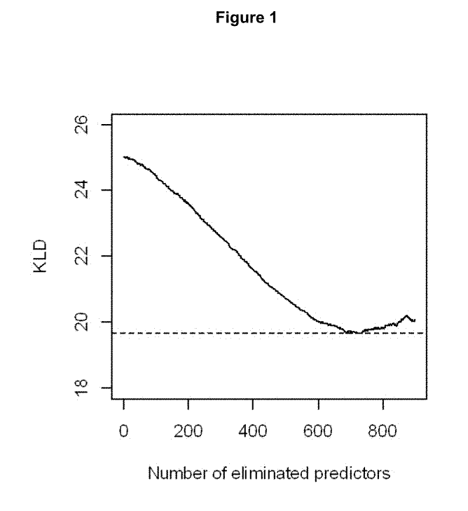

FIG. 1: Backward elimination of potential biomarkers for respiratory sensitization. 1029 genes, selected by p-value sorting, were used as input. After elimination of 727 genes, a local minimum in KLD was observed. Thus, the remaining 302 genes collectively hold the most information relevant for separating respiratory sensitizers from non-sensitizers. This biomarker signature was termed GARD Respiratory Prediction Signature.

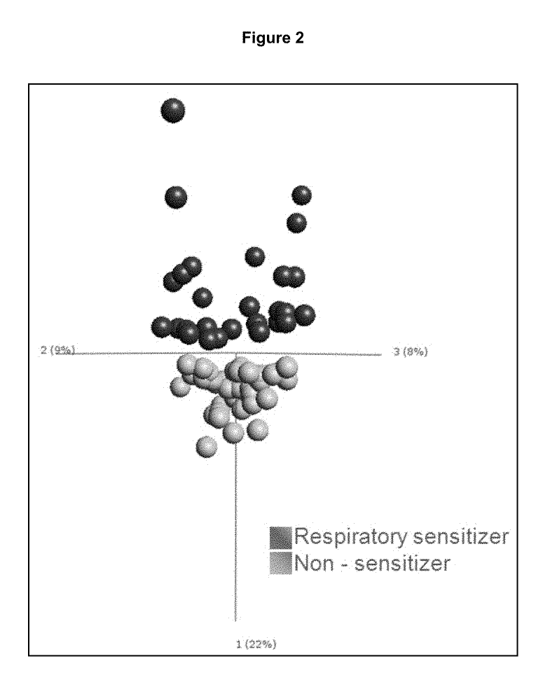

FIG. 2: Principal component analysis based on 302 transcripts chosen by p-value filtering and backward elimination. A complete separation between samples stimulated with respiratory sensitizers (blue) and non-sensitizers (green) is observed.

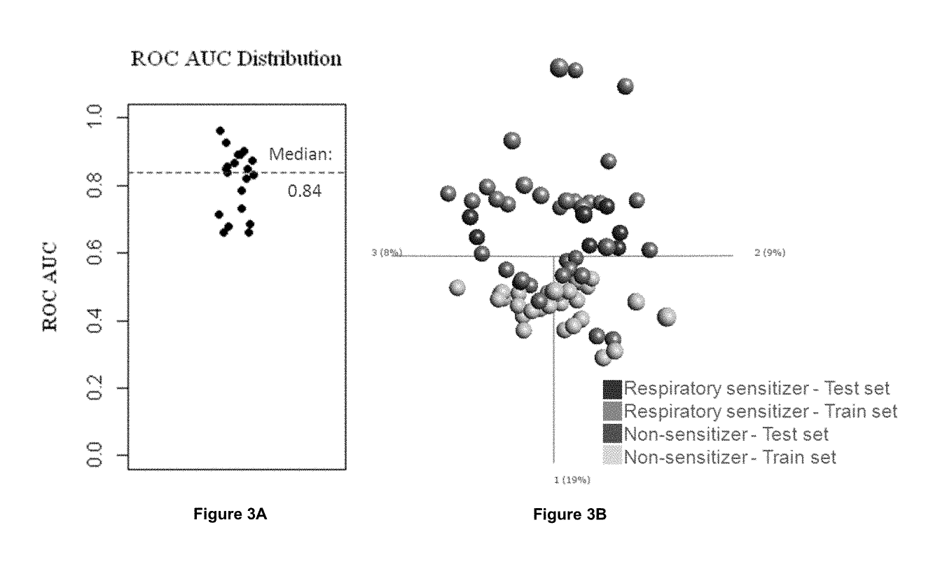

FIGS. 3A and 3B: Estimation of the predictive power of the GARD Respiratory Prediction Signature using cross-validation. 20 Validation Biomarker Signatures were constructed using 70% of randomly chosen data (train set). The Validation Biomarker Signatures were subsequently used to classify the samples in the remaining 30% of the data (test set). FIG. 3A: ROC AUC distribution following SVM predictions of samples in the test set. FIG. 3B: Representative representation of prediction performance illustrated with principal component analysis.

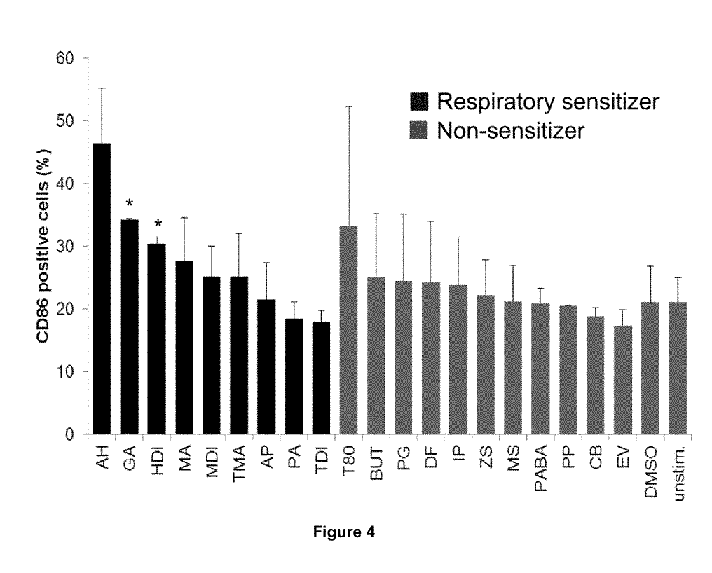

FIG. 4: CD86 expression of MUTZ-3 cells following chemical stimulations. Data shown is an average of (chemical stimulations, n=3, DMSO and unstimulated cells (n=6), with error bars showing standard deviation. Statistical significance was determined by student's t-test, comparing each stimulation with its corresponding vehicle, with p<0.05 indicated by *.

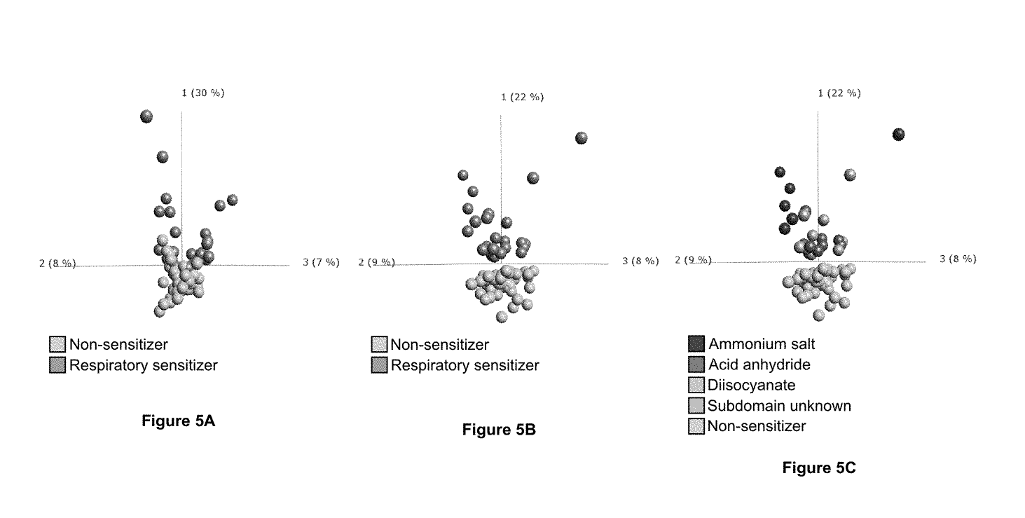

FIGS. 5A, 5B, and 5C: Establishment of a predictive biomarker signature. FIG. 5A: Principal component analysis based on 1029 transcripts chosen by p-value filtering. FIG. 5B: Principal component analysis based on 302 transcripts chosen by Backward Elimination. Samples are colored as respiratory sensitizers (blue, n=27) or non-sensitizers (green, n=47). All data consisting of 74 samples, including all replicates, is represented. FIG. 5C: Respiratory sensitizers are colored according their mechanistic subdomain.

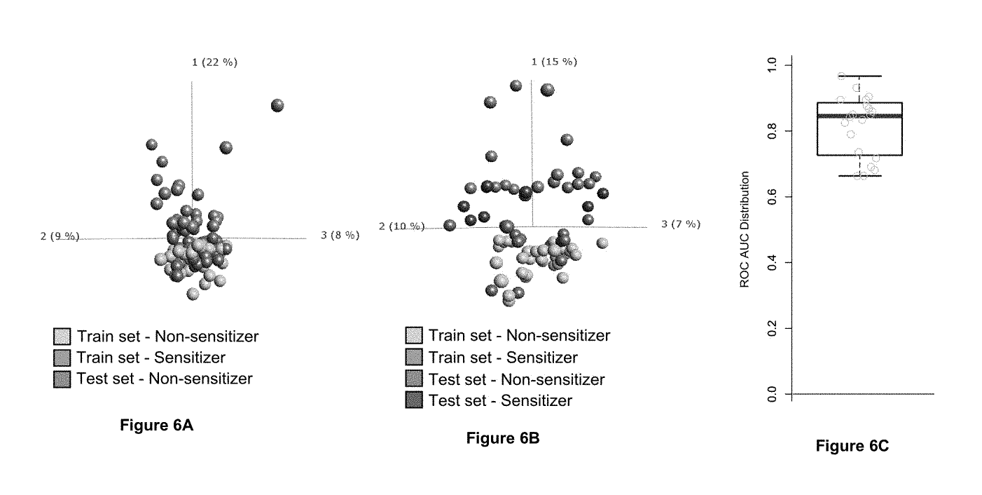

FIGS. 6A, 6B, and 6C: Estimation of the predictive power of the GRPS using an external test set and cross-validation. FIG. 6A: An external data set consisting of triplicates of non-sensitizers were mapped into the PCA space constructed by the GRPS. Only the train data are allowed to influence the principal components. All replicates of samples are represented (train data n=74, test data n=48). FIG. 6B: A Validation Biomarker Signature was constructed using 70% of randomly chosen data (train set). The train set was used to build a PCA space using the Validation Biomarker Signature as a variable input. The remaining 30% of data (test set) was mapped into this space without being allowed to influence the principal components. FIG. 6C: The random division of data into train set and test set were iterated 20 times. The ROC AUC distribution is reported with a box plot, with jittered data points overlaid. The median ROC AUC was 0.84.

### EXAMPLES

### Introduction

Respiratory sensitization to low-molecular weight compounds is a common cause of occupational asthma, which has been associated with fatal outcomes. To prevent the occurrence of respiratory chemical sensitizers and minimize risks in working environments, efforts are being made to develop assays that will predict a compound's′ ability to induce respiratory sensitization. However, to date no validated in vitro or in vivo method, in vitro or in vivo, exists that reliably accomplishes accurate classifications of chemicals as respiratory sensitizers. Recently, we presented a novel in vitro assay for assessment of skin sensitizers, called GARD (Johansson et al., 2011, BMC Genomics, 12:339). We have expanded the applicability of GARD to be able to also classify respiratory sensitizers, using a new genomic biomarker signature set comprising 302 genes associated with immunological events leading to maturation of dendritic cells. Thus, we present an assay with the combined ability to predict both skin and respiratory sensitization ability in assayed compounds.

### Materials and Methods

### Chemicals

A panel of 20 chemical compounds, consisting of 9 respiratory sensitizers and 11 non-sensitizers were used for cell stimulations. The sensitizers were glutaraldehyde, ammonium persulfate, phtalic anhydride, methylene diphenol diisocyanate, ammonium hexachloroplatinate, trimellitic anhydride, hexamethylen diisocyanate, maleic anhydride and toluendiisocyanate. The non-sensitizers were chlorobenzene, zinc sulphate, 4-aminobenzoic acid, methyl salicylate, ethyl vanillin, isopropanol, dimethyl formamide, 1-butanol, potassium permanganate, propylene glycol and tween 80 (Table 2). All chemicals were from Sigma-Aldrich, St. Louis, Mo., USA. Compounds were dissolved in either dimethyl sulfoxide (DMSO) or distilled water. Prior to stimulations, the cytotoxicity of all compounds was monitored, using propidium iodide (PI) (BD Biosciences, San Diego, Calif.) using protocol provided by the manufacturer. The relative viability of stimulated cells was calculated as:

$$Relative viability = \frac{fraction of viable stimulated cells}{fraction of viable unstimulated cells} \cdot 100$$

For toxic compounds, the concentration yielding 90% relative viability (Rv90) was used. For non-toxic compounds, a concentration of 500 μM was used. For non-toxic compounds that were insoluble at 500 μM in medium, the highest soluble concentration was used. For compounds dissolved in DMSO, the final concentration of DMSO in each well was 0.1%. The concentrations used for any given chemical are termed the ‘GARD input concentration’, and are listed in Table 2.

### Chemical Exposure of the Cells

The human myeloid leukemia-derived cell line MUTZ-3 (DSMZ, Braunschweig, Germany) was maintained in α-MEM (Thermo Scientific Hyclone, Logan, Utah) supplemented with 20% (volume/volume) fetal calf serum (Invitrogen, Carlsbad, Calif.) and 40 ng/ml rhGM-CSF (Bayer HealthCare Pharmaceuticals, Seattle, Wash.), as described (Johansson H, Lindstedt M, Albrekt A S, Borrebaeck C A: A genomic biomarker signature can predict skin sensitizers using a cell-based in vitro alternative to animal tests. BMC Genomics 2011, 12:399; Rasaiyaah J, Yong K, Katz D R, Kellam P, Chain B M: Dendritic cells and myeloid leukaemias: plasticity and commitment in cell differentiation. Br J Haematol 2007, 138(3):281-290). Cultures were maintained at 200.000 cells/ml during expansion, with a media change every 3-4 days. No differentiating steps were performed and instead, the proliferating progenitor MUTZ-3 was used for stimulations. Prior to each experiment, the cells were immunophenotyped using flow cytometry as a quality control. Cells were seeded in 6-well plates at 200.000 cells/ml. Stock solutions of each compound were prepared in either DMSO or distilled water, and were subsequently diluted so the in-well concentrations corresponded to the GARD input concentration, and in-well concentrations of DMSO were 0.1%. Cells were incubated for 24 h at 37° C. and 5% CO₂. Thereafter, cells were harvested and analyzed by flow cytometry. In parallel, harvested cells were lysed in TRIzol reagent (Invitrogen) and stored at −20° C. until RNA extraction. Stimulations with chemicals were performed in three individual experiments, so that triplicates samples were obtained.

### Phenotypic Analysis with Flow Cytometry

All cell surface staining and washing steps were performed in PBS containing 1% BSA (w/v). Cells were incubated with specific mouse mAbs for 15 min at 4° C. The following mAbs were used for flow cytometry: FITC-conjugated CD1a (DakoCytomation, Glostrup, Denmark), CD34, CD86, and HLA-DR (BD Biosciences), PE-conjugated CD14 (DakoCytomation), CD54 and CD80 (BD Biosciences). Mouse IgG1, conjugated to FITC or PE were used as isotype controls (BD Biosciences) and PI was used to assess cell viability. FACSDiva software was used for data acquisition with FACSCanto II instrument (BD Bioscience). 10,000 events were acquired and gates were set based on light scatter properties to exclude debris and nonviable cells. Further data analysis was performed using FCS Express V3 (De Novo Software, Los Angeles, Calif.).

### Phenotypic Analysis, Chemical Exposure, Cell Harvest and RNA Isolation

The maintenance and chemical stimulation of MUTZ-3 and all subsequent isolation of RNA and preparation of cDNA was performed as previously described (Johansson H, Albrekt A S, Borrebaeck C A K, Lindstedt M (2012) The GARD assay for assessment of chemical skin sensitizers. Toxicol in Vitro). In short, a phenotypic control of MUTZ-3 was performed prior to chemical stimulation. Stimulated cells were harvested and RNA was isolated. A control of the maturity state of the cells was performed by flow cytometric analysis of CD86. Preparation of cDNA and hybridization, washing and scanning of the Human Gene 1.0 ST Arrays (Affymetrix, Santa Clara, Calif., USA) was performed, according to standardized protocols provided by the manufacturer (Affymetrix).

### Microarray Data Analysis and Statistical Methods

The method by which a predictive signature was established has been previously described (Johansson H, Lindstedt M, Albrekt A S, Borrebaeck C A (2011) A genomic biomarker signature can predict skin sensitizers using a cell-based in vitro alternative to animal tests. BMC Genomics 12: 399). In short, microarray data were normalized and quality checked with the RMA algorithm, using Affymetrix Expression Console (Affymetrix). The top 1029 predictors were selected by p-values from an ANOVA, comparing respiratory sensitizers and non-sensitizers. An algorithm for Backward Elimination (Johansson et al., 2011, supra.; Carlsson A, Wingren C, Kristensson M, Rose C, Ferno M, et al. (2011) Molecular serum portraits in patients with primary breast cancer predict the development of distant metastases. Proc Natl Acad Sci U S A 108: 14252-14257) was applied on the top 1029 predictors, to further reduce the biomarker signature size. The Backward Elimination algorithm was modified to minimize the Kullback-Leibler error (Kullback S, Leibler R A (1951) On Information and Sufficiency. Annals of Mathematical Statistics 22: 79-86) rather than maximizing the Area Under the Receiver Operating Characteristic (ROC AUC) (Lasko T A, Bhagwat J G, Zou K H, Ohno-Machado L (2005) The use of receiver operating characteristic curves in biomedical informatics. J Biomed Inform 38: 404-415), in order to enable continued signature optimization in cases where the ROC AUC reaches 1.0. The selected top 302 predictors were collectively designated “GARD Respiratory Prediction Signature” (GRPS). The script for Backwards Eliminations was programmed in R (R Development Core Team (2008) R: A language and environment for statistical computing. R Foundation for Statistical Computing. Vienna, Austria), with the additional package e1071 (Weingart S N, Iezzoni L I, Davis R B, Palmer R H, Cahalane M, et al. (2000) Use of administrative data to find substandard care: validation of the complications screening program. Med Care 38: 796-806). ANOVA analyses and visualization of results with Principal Component Analysis (PCA) (Ringner M (2008) What is principal component analysis? Nat Biotechnol 26: 303-304) were performed using Qlucore Omics Explorer 2.3 (Qlucore AB, Lund, Sweden). The predictive performance of the GRPS was estimated using an external dataset consisting of negative chemical stimulations, as well as a method for cross-validation based on Support Vector Machines (SVM) (Noble W S (2006) What is a support vector machine? Nat Biotechnol 24: 1565-1567), as described (Johansson et al., 2011, supra.). The biological relevance of the GRPS was explored using Ingenuity Pathways Analysis (IPA) (Ingenuity Systems, Inc. Mountain View, USA), by performing a ‘Core Analysis’. The top 1029 genes were used as IPA input along with fold change values. Biological relevance was established by exploring the Canonical Pathways associated with input molecules. The array data has been uploaded to ArrayExpress (http://www.ebi.ac.uk/arrayexpress/) with accession number E-MEXP-3773.

### Interrogation of the Method for Identification of the Prediction Signature

The data set was divided into a training set and a test set, consisting of 70% and 30%, of the chemical compounds, respectively. The division was performed randomly, while maintaining the proportions of sensitizers and non-sensitizers in each subset at the same ratio as in the complete data set. A test biomarker signature was identified in the training set, using ANOVA filtering and backward elimination, as described above. This test signature was used to train a Support Vector Machine (SVM) (Noble W S: What is a support vector machine? Nat Biotechnol 2006, 24(12):1565-1567), using the training set, which was thereafter applied to predict the samples of the test set. The process was repeated 20 times and the distribution of the area under the Receiver Operating Characteristic (ROC AUC) (Lasko T A, Bhagwat J G, Zou K H, Ohno-Machado L: The use of receiver operating characteristic curves in biomedical informatics. J Biomed Inform 2005, 38(5):404-415) was used as a measurement of the performance of the model.

### Results

### Analysis of the Transcriptional Profiles in Chemically Stimulated MUTZ-3 Cells

Following 24 h stimulations with a panel of reference chemicals, mRNA from MUTZ-3 was collected for transcriptional profiling. The stimulations included 9 different chemical respiratory sensitizers and 11 different non-sensitizers, all sampled in biological triplicates except for 4-aminobenzoic acid, which was sampled in 6 replicates due to internal controls, and potassium permanganate, which was sampled in only 2 replicates due to a faulty array. In addition, DMSO and distilled water was sampled in 6 replicates each, as vehicle controls. Summarized, the dataset ready for analysis consisted of 74 arrays, each with measurements of 29141 transcripts.

The first step of analysis involved a p-value filtering of the genes according to their ability to separate respiratory sensitizers from non-sensitizers, as determined by an ANOVA comparing the two groups. Based on previous experience, approximately 1000 genes is an appropriate amount of potential predictors to use as an input in an algorithm for backward elimination (Johansson H, Lindstedt M, Albrekt A S, Borrebaeck C A: A genomic biomarker signature can predict skin sensitizers using a cell-based in vitro alternative to animal tests. BMC Genomics 2011, 12:399.). Using a p-value cutoff at 0.0067 (FDR 19%), 1029 genes were identified. The backward elimination algorithm was applied, removing the predictor that contributes the least information in an iterative manner. A local minimum in Kullbach-Liebler Divergence (KLD) was observed when 727 predictors was eliminated (FIG. 1). The remaining 302 genes are collectively termed the “GARD Respiratory Prediction Signature”, and their ability to differentiate between respiratory chemical sensitizers and non-sensitizers are illustrated in FIG. 2.

### Interrogation of the Analysis Used to Identify the Prediction Signature

To validate the predictive power of our signature, we used a machine learning method called the Support Vector Machine (SVM) (Noble, 2006, supra.), which maps the data from a training set in space in order to maximize the separation of gene expression induced by sensitizing and non-sensitizing chemicals. As a training set, 70% of the data set was chosen randomly and the entire process of biomarker selection was repeated. Starting with 29,141 transcripts, the signature was reduced to a gene list of equal size as the GARD Respiratory Prediction Signature, i.e. 302 transcripts, termed “Validation Biomarker Signature”, using ANOVA filtering and backward elimination, as described above. An SVM was trained on the train data, using the Validation Biomarker Signature. The trained SVM was then used to classify each sample in the remaining 30% of the data, i.e. the test set, as either a respiratory sensitizer or a non-sensitizer. The performance of the classifications was evaluated with the area under the Receiver Operating Characteristic (ROC AUC). This entire cross-validation was iterated 20 times, each time generating different train and test sets, with each train set yielding different Validation Biomarker Signatures. The results of these cross-validations are illustrated in FIG. 3. The median ROC AUC was found to be 0.84, with a range from 0.66 to 0.96. The large variations in predictive performance imply that the random exclusion of 30% of the data greatly affects the composition of the Validation Biomarker Signature. However, the ability to achieve ROC AUCs of up to 0.96 is strong evidence that when the model is trained on all available data, accurate classifications are indeed possible. This cross-validation demonstrates that the GARD Respiratory Prediction biomarkers are capable of accurately predicting respiratory sensitizing properties of unknown samples.

### MUTZ-3 Phenotype in Unstimulated and Stimulated Cells

Prior to chemical challenge, the cells were quality controlled by measuring the cellular expression of common myeloid and dendritic cell markers using flow cytometry. These markers included CD1a, CD14, CD34, CD54, CD80, CD86 and HLA-DR. No deviations from previously published data were found (Johansson et al., 2011, supra.), ensuring that unstimulated cells were successfully maintained in an immature state. Following chemical stimulation, the general maturity state of the cells was controlled again, as determined by the expression of the co-stimulatory marker CD86, with results presented in FIG. 4. Upregulation of CD86 was evident after a number of chemical stimulations, however, due to large standard deviations, only glutaraldehyde and hexamethylen diisocyanate resulted in statistically significant upregulation of CD86. Furthermore, while not statistically significant, an upregulations of CD86 was also evident after a number of control stimulations. Thus, we concluded that CD86 was an unsuited biomarker for respiratory chemical sensitizers. However, many of the compounds used for stimulations in this study were poorly soluble in cell media, and could not be used in concentrations sufficient to induce cytotoxicity. To this end, the increase of CD86 expression can act as a complementary tool to ensure bioavailability of the chemical stimulations.

### Analysis of the Transcriptional Profiles in Chemically Stimulated MUTZ-3 Cells

Following 24 h stimulations, with a panel of reference chemicals, mRNA from MUTZ-3 was collected for transcriptional profiling. The stimulations included 9 different chemical respiratory sensitizers and 11 different non-sensitizers (negative controls), all analyzed in biological triplicates except for 4-aminobenzoic acid, who was analyzed in 6 replicates due to internal controls, and potassium permanganate, which was analyzed in only 2 replicates due to a faulty array. In addition, DMSO and distilled water was analyzed in 6 replicates each, as vehicle controls. Summarized, the data set ready for analysis consisted of 74 arrays, each with measurements of 29,141 transcripts.

The first step of analysis involved a p-value filtering of the genes, according to their ability to discriminate respiratory sensitizers from non-sensitizers, as determined by an ANOVA comparing the two groups. Due to computational limits, approximately 1000 genes is an appropriate amount of potential predictors to use as an input in the algorithm for Backward Elimination. In the present data set, this pre-selection of predictor candidates resulted in 1029 genes, with a p-value of 0.0067 or lower, with a False Discovery Rate (FDR) (Benjamini Y, Hochberg Y (1995) Controlling the false discovery rate: a practical and powerful approach to multiple testing. Journal of the Royal Statistical Society Series B 57: 289-300) of 19%. Collectively, these genes were able to separate respiratory sensitizers from non-sensitizers. However, a clear separation was not achieved, as illustrated with 3D Principal Component Analysis (PCA) (FIG. 5A). Reducing the number of predictors further, by the ranking given by their p-value, did not achieve a clear separation, even though the data contained predictor candidates with p-values down to 10⁻¹⁰. The Backward Elimination algorithm was then applied, removing the predictors (genes) that contribute the least information. A local minimum in Kullbach-Liebler Divergence (KLD) was observed when 727 predictors were eliminated (data not shown). The remaining 302 genes are collectively termed the “GARD Respiratory Prediction Signature” (GRPS), and their ability to differentiate between respiratory chemical sensitizers and non-sensitizers are illustrated in FIG. 5B. The identities of the genes are listed in Table 1.

Of note, there is a significantly larger variation of transcriptional profiles within the group of respiratory sensitizers, compared to the group of non-sensitizers. A similar phenomenon was observed also when studying skin sensitizers, which was related to the potency of the sensitizer, as well as the propensity of different chemicals to induce different signaling pathways (Johansson et al., 2011, supra.). However, categorically defined sensitizing potency is not available for these respiratory chemical sensitizers (Basketter D A, Kimber I (2011) Assessing the potency of respiratory allergens: uncertainties and challenges. Regul Toxicol Pharmacol 61: 365-372). Instead, we aimed to describe the differences in transcriptional profiles in relation to the mechanistic subdomain of each chemical sensitizer (Enoch S J, Roberts D W, Cronin M T (2010) Mechanistic Category Formation for the Prediction of Respiratory Sensitization. Chem Res Toxicol). FIG. 5C shows the same PCA plot as in FIG. 5B, with sensitizers colored according to their mechanistic subdomain, as listed in Table 2. Ammonium salts tend to be positioned further away from the cluster of non-sensitizers, indicating the most dissimilarity to non-sensitizers in terms of transcriptional profile. However, diisocyanates and acid anhydrides cluster closely together, leaving no possibility to draw any conclusion of any dissimilarities between these two groups at this point. To the best of our knowledge, glutaraldehyde has not been assigned to a mechanistic subdomain, although it groups closely with both acid anhydrides and diisocyanates, and these samples are thus denoted “Subdomain unknown” (FIG. 5C).

### Evaluation of the Predictive Accuracy of the Prediction Signature

The predictive performance of the GRPS was evaluated in two ways. Firstly, an external test set consisting of non-sensitizers was used to confirm their position in a PCA plot, based on the GRPS. Secondly, we used a cross-validation method that randomly divided the data into training and test sets, which then were used to train and evaluate the Support Vector Machine classifications.

The first method was possible to perform due to the availability of an additional set of control chemicals, run in a previous set of experiments in which GARD was first conceived (Johansson et al., 2011, supra.). The compounds in this test set were benzaldehyde, chlorobenzene, diethyl phtalate, glycerol, lactic acid, octanoic acid, phenol, salicylic acid and sodium dodecyl sulphate, all sampled in biological triplicates. In addition, the test set contained nine samples of DMSO and unstimulated controls respectively. FIG. 6A shows the same PCA plot as FIG. 5B, in which the test set has been mapped based on the transcriptional profile of the samples, while not being allowed to influence the principal components. All samples of the test set are correctly grouped together with non-sensitizers of the train set. The lack of respiratory sensitizers in this test set was due to our reluctance to set any of these samples aside, when performing the analysis used to establish the GRPS. Any samples included in this analysis are inappropriate to include in a test set due to the risk of over fitting.

To overcome the problem of having no respiratory sensitizers in a true test set, we used a method for cross-validation. As a training set, 70% of the data set was chosen randomly and the entire process of biomarker selection was repeated. Starting with 29,141 transcripts, the signature was reduced to a gene list of equal size to the GRPS, i.e. 302 transcripts, termed “Validation Biomarker Signature”, using p-value filtering and Backward Elimination, as described above. A Support Vector Machine (SVM) (Noble WS (2006) What is a support vector machine? Nat Biotechnol 24: 1565-1567) was trained on the training data set, using the Validation Biomarker Signature. The trained SVM was then used to classify each sample in the remaining 30% of the data, i.e. the test data set, as either a respiratory sensitizer or a non-sensitizer. The performance of the classifications was evaluated with the area under the Receiver Operating Characteristic (ROC AUC). This entire cross-validation was iterated 20 times, each time generating different training and test sets, with each training set yielding different Validation Biomarker Signatures. The results of these cross-validations are illustrated in FIG. 6B-C. The median ROC AUC was found to be 0.84, with a range from 0.66 to 0.96. In addition, the Validation Call Frequency (VCF) for each gene in the GRPS is listed in Table 1. The VCF describes the frequency by which a certain gene has been included in any of the 20 Validation Biomarker Signatures, thus providing a second measurement by which the predictors can be ranked.

### Canonical Pathways Associated with the GARD Respiratory Prediction Signature

Aiming to investigate the biologic response initiated by respiratory chemical sensitizers in MUTZ-3 cells, the data was analyzed with Ingenuity Pathway Analysis (IPA). The top 1029 genes, selected with p-value filtering, were used as input into IPA, along with values of fold change for each gene. Of the 1029 genes, IPA was able to map 933 to unique IDs. Taking duplicates into account, the dataset ready for IPA analysis consisted of 901 molecules. The primary objective was to elucidate which canonical pathways identified molecules are associated with. Results are listed in Table 1, in order of statistical significance according to IPA.

A clear majority of these identified and significantly regulated pathways are mainly driven by a limited set of molecules. These pathways include TREM1 signaling, altered T cell and B cell signaling in rheumatoid arthritis, communication between adaptive and innate immune cells, B cell development, aryl hydrocarbon receptor signaling, dendritic cell maturation, CD28 signaling in T-helper cells, lipid antigen presentation by CD1, cytotoxic T cell mediated apoptosis of target cells and autoimmune thyroid disease signaling. Of note, central for all of these pathways is the bridge between innate and adaptive immunity, and the engagement of innate immune responses initiated by recognition of foreign substances, leading to dendritic cell maturation. Key aspects of this process that is well monitored by the GRPS include upregulation of innate receptors, such as TLRs and AHR, upregulation of antigen presentation-associated molecules, such as HLA and CD1, upregulation of co-stimulatory molecules, such as CD86 and CD40, and upregulation of proinflammatory effector molecules, such as IL-8 and IL-1B.

### Discussion

A variety of chemicals induce allergic sensitization of not only the skin, but also the respiratory tract, giving rise to occupational asthma and other symptoms (Kimber I, Dearman R J (1997) Cell and molecular biology of chemical allergy. Clin Rev Allergy Immunol 15: 145-168). While not as prevalent as chemicals inducing skin sensitization leading to allergic contact dermatitis, identification and hazard assessment of respiratory chemical sensitizers are equally important, not least due to the severe symptoms, with possible fatal outcomes (Chester D A, Hanna E A, Pickelman B G, Rosenman K D (2005) Asthma death after spraying polyurethane truck bedliner. Am J Ind Med 48: 78-84; Kimber I, Wilks M F (1995) Chemical respiratory allergy. Toxicological and occupational health issues. Hum Exp Toxicol 14: 735-736).

Recently, we presented a cell-based in vitro test method for skin sensitizers, called GARD, which is able to classify chemicals with high accuracy (Johansson et al., 2011, supra.; Johansson H, Albrekt A S, Borrebaeck C A K, Lindstedt M (2012) The GARD assay for assessment of chemical skin sensitizers. Toxicol in Vitro). The assay relies on the transcriptional profiling of MUTZ-3 cells following compound stimulation, using a predefined biomarker signature as readout. As measurements of these biomarkers are based on expression array technology, great opportunities exist to broaden the applicability domain of this assay. In the current study, we present a further development of GARD, allowing for the identification of respiratory chemical sensitizers, using a separate biomarker signature termed GARD Respiratory Prediction Signature (GRPS). The GRPS was identified, using a set of reference chemicals known to be either respiratory sensitizers or non-sensitizers, and identifying differentially expressed genes in these two groups by an ANOVA p-value filtering followed by a feature selection algorithm for Backward Elimination. The intended use of the obtained GRPS will thus be in a combined in vitro assay, in which MUTZ-3 cells are stimulated with unknown compounds to be classified. Using the two distinct biomarker signatures, the compound can be classified as a skin sensitizer, respiratory sensitizer or a non-sensitizer. Chemicals that are able to induce both respiratory and skin sensitization will also be specifically classified as such.

The predictive performance of the assay in classifying respiratory chemical allergens was estimated by two forms of validations. Firstly, an external test set consisting of triplicates of 9 negative stimulations were successfully classified, as shown in FIG. 6A. Secondly, a thorough approach of cross-validation was applied, in which 30% of the data was repeatedly excluded at random to form a test set that were later on classified with an SVM model trained on the remaining 70% of the data. Results of this cross-validation are presented as ROC AUCs, (FIGS. 6B and 6C) with a median of 0.84 in a range from 0.66 to 0.96. The large variations in predictive performance imply that the random exclusion of 30% of the data greatly affects the composition of the Validation Biomarker Signature. Indeed, the variation between different Validation Biomarker Signatures are larger and VCF:s are smaller than expected from previous experience (Johansson et al., 2011, supra.). The impact of the composition of each Validation Biomarker Signature has been investigated, and correlations were considered for among a number of factors such as the presence of certain mechanistic domains in the train set and number of replicates of each stimulation that were removed from each training set. No obvious patterns were revealed that could explain the variations in predictive performance. The deviations are thus believed to depend on the relatively low number of samples used for stimulation, which is in turn a consequence of the scarce number of reference compounds available. However, the ability to achieve ROC AUCs of up to 0.96 display strong evidence that when the model is trained on all available data, accurate classifications are indeed possible.

The current absence of validated or even widely accepted methods for hazard assessment of chemicals inducing respiratory sensitization is in large part due to the lack of understanding of the immunobiological mechanisms by which chemical respiratory sensitization occur (Isola D, Kimber I, Sarlo K, Lalko J, Sipes I G (2008) Chemical respiratory allergy and occupational asthma: what are the key areas of uncertainty? J Appl Toxicol 28: 249-253). Specifically, one of the most elusive issues yet to be resolved is the role of the IgE antibody in allergic sensitization of the respiratory tract to chemicals, and whether there are mechanisms through which such sensitization can be achieved that are independent of IgE antibody (Kimber I, Dearman R J (2002) Chemical respiratory allergy: role of IgE antibody and relevance of route of exposure. Toxicology 181-182: 311-315). There are indeed correlations between IgE antibody levels and clinical symptoms for a number of chemical allergens, e.g. for acid anhydrides. On the contrary, less than half of the patients that are sensitized to diisocyanates demonstrate specific IgE antibody in serum. Still, the consensus opinion is that the relationship between IgE antibody and chemical respiratory allergy is strong (Kimber I, Basketter D A, Gerberick G F, Ryan C A, Dearman R J (2011) Chemical allergy: translating biology into hazard characterization. Toxicol Sci 120 Suppl 1: S238-268). The most convincing argument is that there are technical difficulties in designing probes that successfully detect IgE antibodies specific for chemical haptens. In addition, the time of sampling of blood for allergen-specific IgE in relation to the last time of exposure might influence the outcome of such assays.

In an attempt to monitor and compare the transcriptional profiles of different subtypes of respiratory chemical allergens, FIG. 1C shows a PCA based on the GRPS genes, with chemicals colored according to mechanistic domain. No apparent difference is detectable between diisocyanates and acid anhydrides in this plot, as these two groups cluster closely together. While this does not resolve the issue of possibly different mechanistic pathways in sensitization in vivo, IgE dependent or IgE independent, it does confirm that these groups of chemicals induce similar transcriptional changes in MUTZ-3. Instead, the most extreme transcriptional changes are induced by ammonium salts, such as ammonium hexachloroplatinate and ammonium persulfate. However, the major differences in transcriptional profiles of these two compounds are detectable along the axis of the first principal component, i.e. in the same vectorial direction as sensitizers are separated from non-sensitizers. Thus, we conclude that the GRPS is capable of accurately classifying allergens from various mechanistic subdomains.

To further explore the biological effects of sensitizing chemicals on MUTZ-3, an IPA analysis was performed. In order to achieve sufficient significance in the data, the top 1029 genes from p-value filtering were used as input in the IPA software, rather than the top 302 genes of the GRPS. The IPA output presented in Table 1 lists the canonical signaling pathways with which the top 1029 genes are most significantly associated. A majority of these pathways are mainly driven by a core set of molecules, including CD86, CD40, TLR1, TLR6, various HLA-DR molecules and CD1 molecules. Thus, respiratory chemical sensitizers induce increased antigen presentation and upregulation of co-stimulatory molecules in MUTZ-3, arguably in response to ligation of various pattern recognition receptors (PRRs) and intracellular oxidative stress, as indicated by the significance of aryl hydrocarbon receptor (AHR) signaling and glutathione metabolism.

Taken together, the biologic response in MUTZ-3 to chemical respiratory allergens is dominated by innate immune response signaling pathways that ultimately leads to cell maturation of this dendritic cell model, with enhanced antigen presentation and interaction with other immune cells as the end result. Furthermore, novel findings of usage of signaling pathways that has previously been associated with respiratory sensitization to protein allergens shed some light on the biological process leading to sensitization of the respiratory tract in response to chemical allergens. Thus, the GRPS is indeed relevant in an immunologically mechanistic perspective, and provides measurement of transcripts that monitor the biologic events leading to respiratory sensitization.

In conclusion, we present a predictive biomarker signature for respiratory chemical sensitizers in MUTZ-3 cells that complement the previously described GARD assay for assessment of skin sensitizers. The ability to test for two different endpoints in the same sample provides an attractive and hitherto unique assay for safety assessment of chemicals in an in vitro environment.

### References

1. Boverhof D R, Billington R, Gollapudi B B, Hotchkiss J A, Krieger S M, et al. (2008) Respiratory sensitization and allergy: current research approaches and needs. Toxicol Appl Pharmacol 226: 1-13.

2. Banks D E, Tarlo S M (2000) Important issues in occupational asthma. Curr Opin Pulm Med 6: 37-42.

3. Sastre J, Vandenplas O, Park H S (2003) Pathogenesis of occupational asthma. Eur Respir J 22: 364-373.

4. Zammit-Tabona M, Sherkin M, Kijek K, Chan H, Chan-Yeung M (1983) Asthma caused by diphenylmethane diisocyanate in foundry workers. Clinical, bronchial provocation, and immunologic studies. Am Rev Respir Dis 128: 226-230.

5. Bernstein D I, Patterson R, Zeiss C R (1982) Clinical and immunologic evaluation of trimellitic anhydride-and phthalic anhydride-exposed workers using a questionnaire with comparative analysis of enzyme-linked immunosorbent and radioimmunoassay studies. J Allergy Clin Immunol 69: 311-318.

6. Murdoch R D, Pepys J, Hughes E G (1986) IgE antibody responses to platinum group metals: a large scale refinery survey. Br J Ind Med 43: 37-43.

7. Docker A, Wattie J M, Topping M D, Luczynska C M, Newman Taylor A J, et al. (1987) Clinical and immunological investigations of respiratory disease in workers using reactive dyes. Br J Ind Med 44:534-541.

8. Bourne M S, Flindt M L, Walker J M (1979) Asthma due to industrial use of chloramine. Br Med J 2: 10-12.

9. Verstraelen S, Bloemen K, Nelissen I, Witters H, Schoeters G, et al. (2008) Cell types involved in allergic asthma and their use in in vitro models to assess respiratory sensitization. Toxicol In Vitro 22: 1419-1431.

10. Dearman R J, Basketter D A, Kimber I (1992) Variable effects of chemical allergens on serum IgE concentration in mice. Preliminary evaluation of a novel approach to the identification of respiratory sensitizers. J Appl Toxicol 12: 317-323.

11. Dearman R J, Skinner R A, Humphreys N E, Kimber I (2003) Methods for the identification of chemical respiratory allergens in rodents: comparisons of cytokine profiling with induced changes in serum IgE. J Appl Toxicol 23: 199-207.

12. Verstraelen S, Nelissen I, Hooyberghs J, Witters H, Schoeters G, et al. (2009) Gene profiles of THP-1 macrophages after in vitro exposure to respiratory (non-) sensitizing chemicals: identification of discriminating genetic markers and pathway analysis. Toxicol In Vitro 23: 1151-1162.

13. Verstraelen S, Nelissen I, Hooyberghs J, Witters H, Schoeters G, et al. (2009) Gene profiles of a human bronchial epithelial cell line after in vitro exposure to respiratory (non-)sensitizing chemicals: identification of discriminating genetic markers and pathway analysis. Toxicology 255: 151-159.

14. Verstraelen S, Nelissen I, Hooyberghs J, Witters H, Schoeters G, et al. (2009) Gene profiles of a human alveolar epithelial cell line after in vitro exposure to respiratory (non-)sensitizing chemicals: identification of discriminating genetic markers and pathway analysis. Toxicol Lett 185: 16-22.

15. Lalko J F, Kimber I, Dearman R J, Gerberick G F, Sarlo K, et al. (2011) Chemical reactivity measurements: potential for characterization of respiratory chemical allergens. Toxicol In Vitro 25: 433-445.

16. Johansson H, Lindstedt M, Albrekt A S, Borrebaeck C A (2011) A genomic biomarker signature can predict skin sensitizers using a cell-based in vitro alternative to animal tests. BMC Genomics 12: 399.

17. Johansson H, Albrekt A S, Borrebaeck C A K, Lindstedt M (2012) The GARD assay for assessment of chemical skin sensitizers. Toxicol in Vitro.

18. Santegoets S J, Masterson A J, van der Sluis P C, Lougheed S M, Fluitsma D M, et al. (2006) A CD34(+) human cell line model of myeloid dendritic cell differentiation: evidence for a CD14(+)CD11b(+) Langerhans cell precursor. J Leukoc Biol 80: 1337-1344.

19. Masterson A J, Sombroek C C, De Gruijl T D, Graus Y M, van der Vliet H J, et al. (2002) MUTZ-3, a human cell line model for the cytokine-induced differentiation of dendritic cells from CD34+ precursors. Blood 100: 701-703.

20. Larsson K, Lindstedt M, Borrebaeck C A (2006) Functional and transcriptional profiling of MUTZ-3, a myeloid cell line acting as a model for dendritic cells. Immunology 117: 156-166.

21. Carlsson A, Wingren C, Kristensson M, Rose C, Ferno M, et al. (2011) Molecular serum portraits in patients with primary breast cancer predict the development of distant metastases. Proc Natl Acad Sci U S A 108: 14252-14257.

22. Kullback S, Leibler R A (1951) On Information and Sufficiency. Annals of Mathematical Statistics 22: 79-86.

23. Lasko T A, Bhagwat J G, Zou K H, Ohno-Machado L (2005) The use of receiver operating characteristic curves in biomedical informatics. J Biomed Inform 38: 404-415.

24. R Development Core Team (2008) R: A language and environment for statistical computing. R Foundation for Statistical Computing. Vienna, Austria.

25. Weingart S N, Iezzoni L I, Davis R B, Palmer R H, Cahalane M, et al. (2000) Use of administrative data to find substandard care: validation of the complications screening program. Med Care 38: 796-806.

26. Ringner M (2008) What is principal component analysis? Nat Biotechnol 26: 303-304.

27. Noble W S (2006) What is a support vector machine? Nat Biotechnol 24: 1565-1567.

28. Benjamini Y, Hochberg Y (1995) Controlling the false discovery rate: a practical and powerful approach to multiple testing. Journal of the Royal Statistical Society Series B 57: 289-300.

29. Basketter D A, Kimber I (2011) Assessing the potency of respiratory allergens: uncertainties and challenges. Regul Toxicol Pharmacol 61: 365-372.

30. Enoch S J, Roberts D W, Cronin M T (2010) Mechanistic Category Formation for the Prediction of Respiratory Sensitization. Chem Res Toxicol.

31. Kimber I, Dearman R J (1997) Cell and molecular biology of chemical allergy. Clin Rev Allergy Immunol 15: 145-168.

32. Chester D A, Hanna E A, Pickelman B G, Rosenman K D (2005) Asthma death after spraying polyurethane truck bedliner. Am J Ind Med 48: 78-84.

33. Kimber I, Wilks M F (1995) Chemical respiratory allergy. Toxicological and occupational health issues. Hum Exp Toxicol 14: 735-736.

34. Isola D, Kimber I, Sarlo K, Lalko J, Sipes I G (2008) Chemical respiratory allergy and occupational asthma: what are the key areas of uncertainty? J Appl Toxicol 28: 249-253.

35. Kimber I, Dearman R J (2002) Chemical respiratory allergy: role of IgE antibody and relevance of route of exposure. Toxicology 181-182: 311-315.

36. Kimber I, Basketter D A, Gerberick G F, Ryan C A, Dearman R J (2011) Chemical allergy: translating biology into hazard characterization. Toxicol Sci 120 Suppl 1: S238-268.

TABLE 1
“Core”, “preferred” and “optional” biomarkers
from the GARD Respiratory Prediction Signature.

| | | | |
|---|---|---|---|
| | | Affymetrix | Validation Call |
| Gene Symbol | Entrez Gene ID | Probe Set ID | Frequency |

(A) Core biomarkers

| | | | | |
|---|---|---|---|---|
| 1. | OR5B21 | ENST00000360374 | 7948330 | 100 |
| 2. | SLC7A7 | ENST00000404278 | 7977786 | 95 |

(B) Preferred biomarkers

| | | | | |
|---|---|---|---|---|
| 3. | PIP3-E | ENST00000265198 | 8130408 | 85 |
| 4. | BTNL8 | ENST00000400706 | 8116537 | 85 |
| 5. | CLEC4A | ENST00000360500 | 7953723 | 90 |
| 6. | HIST4H4 | ENST00000358064 | 7961483 | 80 |
| 7. | YKT6 | ENST00000223369 | 8132580 | 80 |
| 8. | FLJ32679 /// | ENST00000327271 | 7981895 | 85 |
| | GOLGA8G /// | | | |
| | GOLGA8E | | | |
| 9. | PACSIN3 | ENST00000298838 | 7947801 | 90 |
| 10. | PDE1B | ENST00000243052 | 7955943 | 80 |
| 11. | NQO1 | ENST00000320623 | 8002303 | 80 |
| 12. | CAMK1D | ENST00000378845 | 7926223 | 95 |
| 13. | MYB | ENST00000341911 | 8122202 | 95 |
| 14. | — | ENST00000387396 | 8065752 | 80 |
| 15. | GRK5 | ENST00000369106 | 7930894 | 90 |

(C) Optional biomarkers

| | | | | |
|---|---|---|---|---|
| 16. | CD86 | ENST00000330540 | 8082035 | 100 |
| 17. | CD1A | ENST00000289429 | 7906339 | 85 |
| 18. | WWOX | ENST00000355860 | 7997352 | 85 |
| 19. | IKZF2 | ENST00000374319 | 8058670 | 85 |
| 20. | FUCA1 | ENST00000374479 | 7913694 | 80 |
| 21. | C10orf76 | ENST00000370033 | 7935951 | 80 |
| 22. | AMICA1 | ENST00000356289 | 7952022 | 80 |
| 23. | PDPK2 /// PDPK1 | ENST00000382326 | 7998825 | 80 |
| 24. | AZU1 | ENST00000334630 | 8024038 | 80 |
| 25. | ACN9 | ENST00000360382 | 8134415 | 80 |
| 26. | PDPN | ENST00000400804 | 7898057 | 75 |
| 27. | LOC642587 | NM_001104548 | 7909422 | 75 |
| 28. | SEC61A2 | ENST00000379051 | 7926189 | 75 |
| 29. | ELA2 | ENST00000263621 | 8024056 | 75 |
| 30. | BMP2K | ENST00000335016 | 8096004 | 75 |
| 31. | HCCS | ENST00000321143 | 8165995 | 75 |
| 32. | CXorf26 | ENST00000373358 | 8168447 | 75 |
| 33. | TYSND1 | ENST00000287078 | 7934114 | 70 |
| 34. | CARS | ENST00000380525 | 7945803 | 70 |
| 35. | NECAP1 | ENST00000339754 | 7953715 | 70 |
| 36. | CDH26 | ENST00000348616 | 8063761 | 70 |
| 37. | SERPINB1 | ENST00000380739 | 8123598 | 70 |
| 38. | STEAP4 | ENST00000301959 | 8140840 | 70 |
| 39. | TXNIP | ENST00000369317 | 7904726 | 65 |
| 40. | — | ENST00000386628 | 7925821 | 65 |
| 41. | C12orf35 | ENST00000312561 | 7954711 | 65 |
| 42. | HMGA2 | ENST00000393578 | 7956867 | 65 |
| 43. | KRT16 | ENST00000301653 | 8015376 | 65 |
| 44. | GGTLC2 | ENST00000215938 | 8071662 | 65 |
| 45. | — | ENST00000386437 | 8089926 | 65 |
| 46. | OSBPL11 | ENST00000393455 | 8090277 | 65 |
| 47. | FAM71F1 | ENST00000315184 | 8135945 | 65 |
| 48. | ATP6V1B2 | ENST00000276390 | 8144931 | 65 |
| 49. | LOC128102 | AF252254 | 7904429 | 60 |
| 50. | TBX19 | ENST00000367821 | 7907146 | 60 |
| 51. | NID1 | ENST00000264187 | 7925320 | 60 |
| 52. | LPXN | ENST00000263845 | 7948332 | 60 |
| 53. | C15orf45 | AK057017 | 7982375 | 60 |
| 54. | RNF111 | ENST00000380504 | 7983953 | 60 |
| 55. | — | ENST00000386861 | 7993183 | 60 |
| 56. | CD33 | ENST00000262262 | 8030804 | 60 |
| 57. | TANK | ENST00000259075 | 8045933 | 60 |
| 58. | ANKRD44 | ENST00000282272 | 8057990 | 60 |
| 59. | WDFY1 | ENST00000233055 | 8059361 | 60 |
| 60. | SDC4 | ENST00000372733 | 8066513 | 60 |
| 61. | TMPRSS11B | ENST00000332644 | 8100701 | 60 |
| 62. | AFF4 | ENST00000265343 | 8114083 | 60 |
| 63. | HBEGF | ENST00000230990 | 8114572 | 60 |
| 64. | XK | ENST00000378616 | 8166723 | 60 |
| 65. | SLAMF7 | ENST00000368043 | 7906613 | 55 |
| 66. | S100A4 | ENST00000368715 | 7920271 | 55 |
| 67. | MPZL3 | ENST00000278949 | 7952036 | 55 |
| 68. | — | GENSCAN00000044853 | 7967586 | 55 |
| 69. | TRAV8-3 | ENST00000390435 | 7973298 | 55 |
| 70. | LOC100131497 | GENSCAN00000046821 | 7980481 | 55 |
| 71. | KIAA1468 | ENST00000299783 | 8021496 | 55 |
| 72. | SPHK2 | ENST00000245222 | 8030078 | 55 |
| 73. | — | ENST00000309260 | 8096554 | 55 |
| 74. | CCR6 | ENST00000283506 | 8123364 | 55 |
| 75. | GSTA3 | ENST00000370968 | 8127087 | 55 |
| 76. | RALA | ENST00000005257 | 8132406 | 55 |
| 77. | C7orf53 | ENST00000312849 | 8135532 | 55 |
| 78. | — | AF480566 | 8141421 | 55 |
| 79. | CERCAM | ENST00000372842 | 8158250 | 55 |
| 80. | — | hsa-mir-147 | 8163729 | 55 |
| 81. | NFYC | ENST00000372655 | 7900468 | 50 |
| 82. | CD53 | ENST00000271324 | 7903893 | 50 |
| 83. | PSEN2 | ENST00000366783 | 7910146 | 50 |
| 84. | CISD1 | ENST00000333926 | 7927649 | 50 |
| 85. | SCD | ENST00000370355 | 7929816 | 50 |
| 86. | MED19 | ENST00000337672 | 7948293 | 50 |
| 87. | SYT17 | ENST00000396244 | 7993624 | 50 |
| 88. | KRT16 /// | ENST00000399124 | 8013465 | 50 |
| | LOC400578 /// | | | |
| | MGC102966 | | | |
| 89. | C18orf51 | ENST00000400291 | 8023864 | 50 |
| 90. | CD79A | ENST00000221972 | 8029136 | 50 |
| 91. | C19orf56 | ENST00000222190 | 8034448 | 50 |
| 92. | AGFG1 | ENST00000409979 | 8048847 | 50 |
| 93. | FOXP1 | ENST00000318796 | 8088776 | 50 |
| 94. | TLR6 | ENST00000381950 | 8099841 | 50 |
| 95. | SUSD3 | ENST00000375472 | 8156393 | 50 |
| 96. | — | ENST00000387842 | 8176921 | 50 |
| 97. | — | ENST00000387842 | 8177424 | 50 |
| 98. | GPA33 | ENST00000367868 | 7922029 | 45 |
| 99. | CDC123 | ENST00000281141 | 7926207 | 45 |
| 100. | C10orf11 | ENST00000354343 | 7928534 | 45 |
| 101. | — | ENST00000322493 | 7937971 | 45 |
| 102. | PTMAP7 | AF170294 | 7976239 | 45 |
| 103. | ARRDC4 | ENST00000268042 | 7986350 | 45 |
| 104. | — | ENST00000388199 | 7997738 | 45 |
| 105. | — | ENST00000388437 | 8009299 | 45 |
| 106. | KRT9 | ENST00000246662 | 8015357 | 45 |
| 107. | — | ENST00000379371 | 8035868 | 45 |
| 108. | HDAC4 | ENST00000345617 | 8060030 | 45 |
| 109. | CD200 | ENST00000315711 | 8081657 | 45 |
| 110. | PAPSS1 | ENST00000265174 | 8102214 | 45 |
| 111. | ORAI2 | ENST00000356387 | 8135172 | 45 |
| 112. | — | AK124536 | 8144569 | 45 |
| 113. | ZBTB10 | ENST00000379091 | 8147040 | 45 |
| 114. | — | ENST00000387422 | 8159963 | 45 |
| 115. | RAB9A | ENST00000243325 | 8166098 | 45 |
| 116. | — | — | 7895613 | 40 |
| 117. | DRD5 | ENST00000304374 | 7905025 | 40 |
| 118. | CNR2 | ENST00000374472 | 7913705 | 40 |
| 119. | OIT3 | ENST00000334011 | 7928330 | 40 |
| 120. | — | ENST00000386981 | 7933008 | 40 |
| 121. | C10orf90 | ENST00000356858 | 7936996 | 40 |
| 122. | OR52D1 | ENST00000322641 | 7938008 | 40 |
| 123. | ZNF214 | ENST00000278314 | 7946288 | 40 |
| 124. | — | ENST00000386959 | 7954690 | 40 |
| 125. | ART4 | ENST00000228936 | 7961507 | 40 |
| 126. | RCBTB2 | ENST00000344532 | 7971573 | 40 |
| 127. | HOMER2 | ENST00000304231 | 7991034 | 40 |
| 128. | WWP2 | ENST00000359154 | 7996976 | 40 |
| 129. | WDR24 | ENST00000248142 | 7998280 | 40 |
| 130. | MED31 | ENST00000225728 | 8011968 | 40 |
| 131. | CALM2 | ENST00000272298 | 8052010 | 40 |
| 132. | DLX2 | ENST00000234198 | 8056784 | 40 |
| 133. | BTBD3 | ENST00000399006 | 8060988 | 40 |
| 134. | — | ENST00000339367 | 8075817 | 40 |
| 135. | TBCA | ENST00000380377 | 8112767 | 40 |
| 136. | GIN1 | ENST00000399004 | 8113403 | 40 |
| 137. | NOL7 | ENST00000259969 | 8116969 | 40 |
| 138. | — | ENST00000402365 | 8117628 | 40 |
| 139. | C7orf28B /// | ENST00000325974 | 8138128 | 40 |
| | C7orf28A | | | |
| 140. | DPP7 | ENST00000371579 | 8165438 | 40 |
| 141. | hCG_1749005 | NR_003933 | 8167640 | 40 |
| 142. | PNPLA4 | ENST00000381042 | 8171229 | 40 |
| 143. | USP51 | ENST00000330856 | 8173174 | 40 |
| 144. | HLA-DQA1 /// | ENST00000383127 | 8178193 | 40 |
| | HLA-DRA | | | |
| 145. | FAAH | ENST00000243167 | 7901229 | 35 |
| 146. | GDAP2 | ENST00000369443 | 7918955 | 35 |
| 147. | CD48 | ENST00000368046 | 7921667 | 35 |
| 148. | PTPRJ | ENST00000278456 | 7939839 | 35 |
| 149. | EXPH5 | ENST00000265843 | 7951545 | 35 |
| 150. | RPS26 /// | ENST00000393490 | 7956114 | 35 |
| | LOC728937 /// | | | |
| | RPS26L /// | | | |
| | hCG_2033311 | | | |
| 151. | ALDH2 | ENST00000261733 | 7958784 | 35 |
| 152. | CALM1 | ENST00000356978 | 7976200 | 35 |
| 153. | NOX5 /// SPESP1 | ENST00000395421 | 7984488 | 35 |
| 154. | RHBDL1 | ENST00000352681 | 7992010 | 35 |
| 155. | CYLD | ENST00000311559 | 7995552 | 35 |
| 156. | OSBPL1A | ENST00000357041 | 8022572 | 35 |
| 157. | GYPC | ENST00000259254 | 8045009 | 35 |
| 158. | RQCD1 | ENST00000295701 | 8048340 | 35 |
| 159. | RBM44 | ENST00000316997 | 8049552 | 35 |
| 160. | — | ENST00000384680 | 8051862 | 35 |
| 161. | C3orf58 | ENST00000315691 | 8083223 | 35 |
| 162. | MFSD1 | ENST00000264266 | 8083656 | 35 |
| 163. | HACL1 | ENST00000321169 | 8085608 | 35 |
| 164. | SATB1 | ENST00000338745 | 8085716 | 35 |
| 165. | USP4 | ENST00000351842 | 8087380 | 35 |
| 166. | — | ENST00000410125 | 8089928 | 35 |
| 167. | — | ENST00000384055 | 8097445 | 35 |
| 168. | IL7R | ENST00000303115 | 8104901 | 35 |
| 169. | — | ENST00000364497 | 8117018 | 35 |
| 170. | FAM135A | ENST00000370479 | 8120552 | 35 |
| 171. | CD164 | ENST00000310786 | 8128716 | 35 |
| 172. | DYNLT1 | ENST00000367088 | 8130499 | 35 |
| 173. | NRCAM | ENST00000379027 | 8142270 | 35 |
| 174. | ZNF596 | ENST00000308811 | 8144230 | 35 |
| 175. | — | ENST00000332418 | 8170322 | 35 |
| 176. | TCEAL3 /// TCEAL6 | ENST00000372774 | 8174134 | 35 |
| 177. | SNAPIN | ENST00000368685 | 7905598 | 30 |
| 178. | DENND2D | ENST00000369752 | 7918487 | 30 |
| 179. | SAMD8 | ENST00000372690 | 7928516 | 30 |
| 180. | LHPP | ENST00000368842 | 7931204 | 30 |
| 181. | SLC37A2 | ENST00000298280 | 7944931 | 30 |
| 182. | FLI1 /// EWSR1 | ENST00000344954 | 7945132 | 30 |
| 183. | OR9G4 | ENST00000395180 | 7948157 | 30 |
| 184. | LOC338799 | ENST00000391388 | 7967210 | 30 |
| 185. | HEXDC | ENST00000337014 | 8010787 | 30 |
| 186. | NOTUM | ENST00000409678 | 8019334 | 30 |
| 187. | MCOLN1 | ENST00000394321 | 8025183 | 30 |
| 188. | PRKACA | ENST00000350356 | 8034762 | 30 |
| 189. | CRIM1 | ENST00000280527 | 8041447 | 30 |
| 190. | CECR5 | ENST00000336737 | 8074227 | 30 |
| 191. | RNF13 | ENST00000392894 | 8083310 | 30 |
| 192. | 40969 | ENST00000339875 | 8103508 | 30 |
| 193. | ZNF366 | ENST00000318442 | 8112584 | 30 |
| 194. | — | ENST00000410754 | 8120979 | 30 |
| 195. | GIMAP5 | ENST00000358647 | 8137257 | 30 |
| 196. | — | ENST00000362484 | 8147242 | 30 |
| 197. | TFE3 | ENST00000315869 | 8172520 | 30 |
| 198. | RHOU | ENST00000366691 | 7910387 | 25 |
| 199. | MED8 | ENST00000290663 | 7915516 | 25 |
| 200. | CASQ2 | ENST00000261448 | 7918878 | 25 |
| 201. | NUDT5 | ENST00000378940 | 7932069 | 25 |
| 202. | C11orf73 | ENST00000278483 | 7942932 | 25 |
| 203. | PAK1 | ENST00000356341 | 7950578 | 25 |
| 204. | PRSS21 | ENST00000005995 | 7992722 | 25 |
| 205. | — | ENST00000332418 | 7997907 | 25 |
| 206. | BTBD12 | ENST00000294008 | 7999008 | 25 |
| 207. | DHRS13 | ENST00000394901 | 8013804 | 25 |
| 208. | CCDC102B | ENST00000319445 | 8021685 | 25 |
| 209. | BCL2 | ENST00000398117 | 8023646 | 25 |
| 210. | ZNF211 /// ZNF134 | ENST00000396161 | 8031784 | 25 |
| 211. | NDUFV2 | ENST00000340013 | 8039068 | 25 |
| 212. | MYCN | ENST00000281043 | 8040419 | 25 |
| 213. | — | ENST00000385528 | 8045561 | 25 |
| 214. | — | ENST00000362957 | 8046522 | 25 |
| 215. | CASP8 | ENST00000264275 | 8047419 | 25 |
| 216. | RTN4 | ENST00000394611 | 8052204 | 25 |
| 217. | PLCG1 | ENST00000244007 | 8062623 | 25 |
| 218. | MGC42105 | ENST00000326035 | 8105146 | 25 |
| 219. | EMB | ENST00000303221 | 8112007 | 25 |
| 220. | — | ENST00000386433 | 8121249 | 25 |
| 221. | COL21A1 | ENST00000370817 | 8127201 | 25 |
| 222. | LRP12 | ENST00000276654 | 8152280 | 25 |
| 223. | LMNA | ENST00000368301 | 7906085 | 20 |
| 224. | — | ENST00000385567 | 7907535 | 20 |
| 225. | — | ENST00000362863 | 7926805 | 20 |
| 226. | ZNF503 | ENST00000372524 | 7934553 | 20 |
| 227. | NLRX1 | ENST00000397884 | 7944463 | 20 |
| 228. | — | ENST00000391173 | 7954775 | 20 |
| 229. | NDRG2 | ENST00000298687 | 7977621 | 20 |
| 230. | TRAF7 | ENST00000326181 | 7992529 | 20 |
| 231. | KRT40 | ENST00000400879 | 8015152 | 20 |
| 232. | KRT40 | ENST00000400879 | 8019604 | 20 |
| 233. | DRD5 | ENST00000304374 | 8053725 | 20 |
| 234. | ZC3H8 | ENST00000409573 | 8054664 | 20 |
| 235. | MMP9 | ENST00000372330 | 8063115 | 20 |
| 236. | PLTP | ENST00000372420 | 8066619 | 20 |
| 237. | — | ENST00000362686 | 8100476 | 20 |
| 238. | SPEF2 | ENST00000282469 | 8104856 | 20 |
| 239. | LRRC16A | ENST00000332168 | 8117243 | 20 |
| 240. | FBXO9 | AK095315 | 8120269 | 20 |
| 241. | EEPD1 | ENST00000242108 | 8132305 | 20 |
| 242. | FCN1 | ENST00000371807 | 8165011 | 20 |
| 243. | EFNA3 | ENST00000368408 | 7905918 | 15 |
| 244. | — | ENST00000314893 | 7910385 | 15 |
| 245. | TMEM19 | ENST00000266673 | 7957167 | 15 |
| 246. | PLXNC1 | ENST00000258526 | 7957570 | 15 |
| 247. | NHLRC3 | ENST00000379599 | 7968703 | 15 |
| 248. | MBNL2 | ENST00000397601 | 7969677 | 15 |
| 249. | EIF5 | ENST00000216554 | 7977058 | 15 |
| 250. | PLEKHG4 | ENST00000360461 | 7996516 | 15 |
| 251. | COPS3 | ENST00000268717 | 8013094 | 15 |
| 252. | FAM171A2 | ENST00000398346 | 8016033 | 15 |
| 253. | LOC653653 /// AP1S2 | ENST00000380291 | 8017210 | 15 |
| 254. | VAPA | ENST00000340541 | 8020129 | 15 |
| 255. | MATK | ENST00000395040 | 8032682 | 15 |
| 256. | ACTR2 | ENST00000377982 | 8042337 | 15 |
| 257. | BPI | ENST00000262865 | 8062444 | 15 |
| 258. | ERG | ENST00000398905 | 8070297 | 15 |
| 259. | LAMB2 | ENST00000305544 | 8087337 | 15 |
| 260. | — | BC090058 | 8133752 | 15 |
| 261. | PHTF2 | ENST00000248550 | 8133818 | 15 |
| 262. | — | ENST00000333261 | 8133902 | 15 |
| 263. | C8orf55 | ENST00000336138 | 8148559 | 15 |
| 264. | PDE7A | ENST00000379419 | 8151074 | 15 |
| 265. | NAPRT1 | ENST00000340490 | 8153430 | 15 |
| 266. | HLA-DRA | ENST00000383127 | 8179481 | 15 |
| 267. | SLC22A15 | ENST00000369503 | 7904226 | 10 |
| 268. | FCGR1A /// | ENST00000369384 | 7905047 | 10 |
| | FCGR1B /// | | | |
| | FCGR1C | | | |
| 269. | SLC27A3 | ENST00000271857 | 7905664 | 10 |
| 270. | ID3 | ENST00000374561 | 7913655 | 10 |
| 271. | TBCEL | ENST00000284259 | 7944623 | 10 |
| 272. | FAM138D | ENST00000355746 | 7960172 | 10 |
| 273. | POMP | ENST00000380842 | 7968297 | 10 |
| 274. | SNN | ENST00000329565 | 7993259 | 10 |
| 275. | MED13 | ENST00000262436 | 8017312 | 10 |
| 276. | ZFP36L2 | ENST00000282388 | 8051814 | 10 |
| 277. | UXS1 | ENST00000409501 | 8054395 | 10 |
| 278. | CD40 | ENST00000279061 | 8063156 | 10 |
| 279. | — | ENST00000362620 | 8066960 | 10 |
| 280. | GGT5 | ENST00000327365 | 8074991 | 10 |
| 281. | — | BC035666 | 8103023 | 10 |
| 282. | G6PD | ENST00000393562 | 8176133 | 10 |
| 283. | — | ENST00000384272 | 7902365 | 5 |
| 284. | CLCC1 | ENST00000369971 | 7918255 | 5 |
| 285. | SCGB2A1 | ENST00000244930 | 7940626 | 5 |
| 286. | GAA | ENST00000302262 | 8010354 | 5 |
| 287. | SERPINB2 | ENST00000404622 | 8021635 | 5 |
| 288. | GPI | ENST00000356487 | 8027621 | 5 |
| 289. | LASS6 | ENST00000392687 | 8046086 | 5 |
| 290. | EIF4A2 | AB209021 | 8084704 | 5 |
| 291. | HLA-DRA | ENST00000383127 | 8118548 | 5 |
| 292. | — | ENST00000385586 | 8136889 | 5 |
| 293. | ANXA2P2 | M62898 /// | 8154836 | 5 |
| | | NR_003573 | | |
| 294. | FANCG | ENST00000378643 | 8160935 | 5 |
| 295. | FAM53B | ENST00000337318 | 7936884 | 0 |
| 296. | RFXAP | ENST00000255476 | 7968653 | 0 |
| 297. | UBR1 | ENST00000382177 | 7987981 | 0 |
| 298. | TBC1D2B | ENST00000409931 | 7990657 | 0 |
| 299. | SERPINB10 | ENST00000397996 | 8021645 | 0 |
| 300. | SEC23B | ENST00000377481 | 8061186 | 0 |
| 301. | MN1 | ENST00000302326 | 8075126 | 0 |
| 302. | CRTAP | ENST00000320954 | 8078450 | 0 |

List of potential predictor genes for respiratory chemical sensitization, identified by ANOVA and backward elimination. Genes are annotated with Entrez Gene ID where found (www.ncbi.nlm.nih.gov/gene). The Affymetrix Probe Set ID for the Human ST 1.0 Array are provided. The validation call frequency (%) is the occurrence of each gene in the 20 Validation Biomarker Signatures obtained during cross-validation.

TABLE 2
Concentrations and vehicles used for each reference chemical.

| | | | | | |
|---|---|---|---|---|---|
| | | | Max solubility | Rv90 | GARD input |
| Compound | Abbreviation | Vehicle | (μM) | (μM) | concentration (μM) |

Respiratory sensitizers

| | | | | | |
|---|---|---|---|---|---|
| Ammonium hexachloroplatinate | AH | Water | 35 | — | 35 |
| Ammonium persulfate | AP | DMSO | — | — | 500 |
| Glutaraldehyde | GA | Water | — | 10 | 10 |
| Hexamethylen diisocyanate | HDI | DMSO | 100 | — | 100 |
| Maleic Anhydride | MA | DMSO | — | — | 500 |
| Methylene diphenol diisocyanate | MDI | DMSO | 50 | — | 50 |
| Phtalic Anhydride | PA | DMSO | 200 | — | 200 |
| Toluendiisocyanate | TDI | DMSO | 40 | — | 40 |
| Trimellitic anhydride | TMA | DMSO | 150 | — | 150 |

Non-sensitizers

| | | | | | |
|---|---|---|---|---|---|
| 1-Butanol | BUT | DMSO | — | — | 500 |
| 4-Aminobenzoic acid | PABA | DMSO | — | — | 500 |
| Chlorobenzene | CB | DMSO | 98 | — | 98 |
| Dimethyl formamide | DF | Water | — | — | 500 |
| Ethyl vanillin | EV | DMSO | — | — | 500 |
| Isopropanol | IP | Water | — | — | 500 |
| Methyl salicylate | MS | DMSO | — | — | 500 |
| Propylene glycol | PG | Water | — | — | 500 |
| Potassium permanganate | PP | Water | 38 | — | 38 |
| Tween 80 | T80 | DMSO | — | — | 500 |
| Zinc sulphate | ZS | Water | 126 | — | 126 |

List of concentrations and vehicles used for each reference chemical used for assay development.
Reference chemicals were classified as respiratory sensitizers or non-respiratory sensitizers through clinical observations in humans.

TABLE 4
Canonical Pathways associated with GRPS.

| Canonical Pathway | -log(p-value) | Regulated molecules¹ |
|---|---|---|
| TREM1 Signaling | 5.4 | CASP1, CCL2, CCL3, CD40, CD86, FCGR2B, |
| | | IL8, IL1B, MPO, PLCG1, SIGIRR, TLR1, TLR6 |
| Altered T Cell and B Cell | 3.7 | CD40, CD86, CD79A, FAS, FCER1G, HLA- |
| Signaling in Rheumatoid | | DQA1, HLA-DRA, IL1B, IL1RN, PRTN3, SPP1, |
| Arthritis | | TLR1, TLR6 |
| Nicotinate and Nicotinamide | 3.6 | CD38, CDK6, DFFB, ENPP2, GRK5, MAP2K1, |
| Metabolism | | MAPK6, NADK, NAPRT1, NNT, PAK1, PPM1F, |
| | | PTPRJ, PTPRO, SGK1 |
| Communication between | 2.9 | CCL3, CD40, CD86, FCER1G, HLA-DRA, IFNA5, |
| Adaptive and Innate Immune | | IL8, IL1B, IL1RN, TLR1, TLR6 |
| Cells | | |
| B Cell Development | 2.9 | CD40, CD86, CD79A, HLA-DQA1, HLA-DRA, |
| | | IL7R |
| Sphingolipid Metabolism | 2.6 | ASAH2, CERK, CERS6, FUT4, KDSR, NAAA, |
| | | PPM1F, PTPRJ, PTPRO, SPHK2, SPTLC2 |
| Cell Cycle Control of | 2.6 | CDK6, CDT1, MCM2, MCM4, MCM6, MCM7 |
| Chromosomal Replication | | |
| Riboflavin Metabolism | 2.6 | ACPP, ENPP2, PPM1F, PTPRJ, PTPRO |
| Glutathione Metabolism | 2.5 | G6PD, GGT5, GGTLC2, GLRX, GSTA3, H6PD, |
| | | IDH2, MGST1, |
| Aryl Hydrocarbon Receptor | 2.4 | AHR, ALDH1A1, CDK6, CDKN1A, CYP1B1, FAS, |
| Signaling | | GSTA3, IL1B, JUN, MCM7, MGST1, NCOA3, |
| | | NQO1, NQO2, RB1 |
| Graft-versus-Host Defense | 2.3 | CD86, FAS, FCER1G, HLA-DQA1, HLA-DRA, |
| Signaling | | IL1B, IL1RN |
| Dendritic Cell Maturation | 2.3 | CD40, CD86, CD1A, CD1B, CD1C, CREB3L4, |
| | | FCER1G, FCGR2A, FCGR2B, HLA-DQA1, HLA-DRA, |
| | | IFNA5, IL1B, IL1RN, MAPK12, PIK3CD, |
| | | PLCG1 |
| CD28 Signaling in T-Helper | 2.3 | ACTR2, CALM1, CD86, FCER1G, HLA-DQA1, |
| Cells | | HLA-DRA, JUN, MAP2K1, MAPK12, PAK1, |
| | | PDPK1, PIK3CD, PLCG1 |
| Lipid Antigen Presentation | 2.3 | CD1A, CD1B, CD1C, FCER1G |
| by CD1 | | |
| Cytotoxic T Cell Mediated | 2.2 | BCL2, CASP8, DFFB, FAS, FCER1G, HLA- |
| Apoptosis of Target Cells | | DQA1, HLA-DRA |
| Fatty Acid Biosynthesis | 2.1 | ACACA, FASN, SLC27A3 |
| Autoimmune Thyroid | 2.0 | CD40, CD86, FAS, FCER1G, HLA-DQA1, HLA-DRA |
| Disease Signaling | | |

¹Molecules indicated in bold are present in the GARD Respiratory Prediction Signature. Molecules colored red are up

Table 4 Legend. Top Canonical Pathways associated with the top 1029 predictors able to separate respiratory chemical sensitizers from non-sensitizers. Molecules indicated in bold are present in the GRPS. Molecules colored red are up regulated in chemical respiratory sensitizers, while molecules colored green are down regulated in chemical respirator sensitizers.

1. A method for identifying agents capable of inducing respiratory sensitization in a mammal comprising or consisting of the steps of:
a) exposing a population of dendritic cells or a population of dendritic-like cells to a test agent; and
b) measuring in the cells the expression of one or more biomarker(s) selected from the group defined in Table 1A and/or Table 1B;
wherein the expression in the cells of the one or more biomarkers measured in step (b) is indicative of the sensitizing effect of the sample to be tested.

2. The method according to claim 1 further comprising:
c) exposing a separate population of the dendritic cells or dendritic-like cells to one or more negative control agent that is not a respiratory sensitizer in a mammal; and
d) measuring in the cells the expression of the one or more biomarker(s) measured in step (b)
wherein the test agent is identified as a respiratory sensitizer in the event that the presence and/or amount in the test sample of the one or more biomarker measured in step (d) differs from the presence and/or amount in the control sample of the one or more biomarker measured in step (b).

3. The method according to claim 1 or 2 further comprising:
e) exposing a separate population of the dendritic cells or dendritic-like cells to one or more positive control agent that is a respiratory sensitizer in a mammal; and
f) measuring in the cells the expression of the one or more biomarker(s) measured in step (b)
wherein the test agent is identified as a respiratory sensitizer in the event that the presence and/or amount in the test sample of the one or more biomarker measured in step (f) corresponds to the presence and/or amount in the positive control sample of the one or more biomarker measured in step (b).

4. The method according to any one of the preceding claims wherein step (b) comprises or consists of measuring the expression of one or more biomarkers defined in Table 1A, for example, at least 2 of the biomarkers defined in Table 1A.

5. The method according to any one of the preceding claims wherein step (b) comprises or consists of measuring the expression of OR5B21.

6. The method according to any one of the preceding claims wherein step (b) comprises or consists of measuring the expression of SLC7A7.

7. The method according to any one of the preceding claims wherein step (b) comprises or consists of measuring the expression of OR5B21 and SLC7A7.

8. The method according to any one of the preceding claims wherein step (b) comprises or consists of measuring the expression of one or more of the biomarkers defined in Table 1B, for example, 2, 3, 4, 5, 6, 7, 8, 9, 10, 11, 12 or 13 of the biomarkers defined in Table 1B.

9. The method according to any one of the preceding claims wherein step (b) comprises or consists of measuring the expression of all of the biomarkers defined in Table 1B.

10. The method according to any one of the preceding claims wherein step (b) comprises or consists of measuring the expression of one or more of the biomarkers defined in Table 10, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286 or 287 of the biomarkers defined in Table 10.

11. The method according to any one of the preceding claims wherein step (b) comprises or consists of measuring the expression of all of the biomarkers defined in Table 1C.

12. The method according to any one of the preceding claims wherein step (b) comprises or consists of measuring the expression of all of the biomarkers defined in Table 1.

13. The method according to any one of the preceding claims wherein step (b) comprises measuring the expression of a nucleic acid molecule encoding the one or more biomarker(s).

14. The method according to claim 13 wherein the nucleic acid molecule is a cDNA molecule or an mRNA molecule.

15. The method according to claim 13 wherein the nucleic acid molecule is an mRNA molecule.

16. The method according to claim 13 wherein the nucleic acid molecule is an cDNA molecule.

17. The method according to any one of claims 13 to 16 wherein measuring the expression of the one or more biomarker(s) in step (b) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

18. The method according to any one of claims 13 to 17 wherein measuring the expression of the one or more biomarker(s) in step (b) is determined using a DNA microarray.

19. The method according to any one of the preceding claims wherein measuring the expression of the one or more biomarker(s) in step (b) is performed using one or more binding moieties, each capable of binding selectively to a nucleic acid molecule encoding one of the biomarkers identified in Table 1.

20. The method according to claim 19 wherein the one or more binding moieties each comprise or consist of a nucleic acid molecule.

21. The method according to claim 20 wherein the one or more binding moieties each comprise or consist of DNA, RNA, PNA, LNA, GNA, TNA or PMO.

22. The method according to claim 19 or 20 wherein the one or more binding moieties each comprise or consist of DNA.

23. The method according to any one of claims 20 to 23 wherein the one or more binding moieties are 5 to 100 nucleotides in length.

24. The method according to any one of claims 20 24 wherein the one or more nucleic acid molecules are 15 to 35 nucleotides in length.

25. The method according to any one of claims 20 to 24 wherein the binding moiety comprises a detectable moiety.

26. The method according to claim 25 wherein the detectable moiety is selected from the group consisting of: a fluorescent moiety; a luminescent moiety; a chemiluminescent moiety; a radioactive moiety (for example, a radioactive atom); or an enzymatic moiety.

27. The method according to claim 26 wherein the detectable moiety comprises or consists of a radioactive atom.

28. The method according to claim 27 wherein the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

29. The method according to claim 26 wherein the detectable moiety of the binding moiety is a fluorescent moiety.

30. The method according to any one of claims 1 to 21 wherein step (b) comprises or consists of measuring the expression of the protein of the one or more biomarker defined in step (b).

31. The method according to claim 30 wherein measuring the expression of the one or more biomarker(s) in step (b) is performed using one or more binding moieties each capable of binding selectively to one of the biomarkers identified in Table 1.

32. The method according to claim 31 wherein the one or more binding moieties comprise or consist of an antibody or an antigen-binding fragment thereof.

33. The method according to claim 32 wherein the antibody or fragment thereof is a monoclonal antibody or fragment thereof.

34. The method according to claim 32 or 33 wherein the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]).

35. The method according to claim 34 wherein the antibody or antigen-binding fragment is a single chain Fv (scFv).

36. The method according to claim 31 wherein the one or more binding moieties comprise or consist of an antibody-like binding agent, for example an affibody or aptamer.

37. The method according to any one of claims 31 to 36 wherein the one or more binding moieties comprise a detectable moiety.

38. The method according to claim 37 wherein the detectable moiety is selected from the group consisting of a fluorescent moiety, a luminescent moiety, a chemiluminescent moiety, a radioactive moiety and an enzymatic moiety.

39. The method according to any one of the preceding claims wherein step (b) is performed using an array.

40. The method according to claim 39 wherein the array is a bead-based array.

41. The method according to claim 40 wherein the array is a surface-based array.

42. The method according to any one of claims 39 to 41 wherein the array is selected from the group consisting of: macroarray; microarray; nanoarray.

43. An array for use in a method according any one of the preceding claims, the array comprising one or more first binding agents as defined in any one of claims 19 to 29 and 31 to 38.

44. An array according to claim 43 comprising binding agents which are collectively capable of binding to all of the biomarkers defined in Table 1.

45. An array according to claim 43 or 44 wherein the first binding agents are immobilised.

46. The method according to any one of the preceding claims for identifying agents capable of inducing a respiratory hypersensitivity response.

47. The method according to any one of the preceding claims wherein the hypersensitivity response is a humoral hypersensitivity response.

48. The method according to claim 46 or 47 wherein the hypersensitivity response is a type I hypersensitivity response.

49. The method according to any one of the preceding claims for identifying agents capable of inducing respiratory allergy.

50. The method according to any one of the preceding claims wherein the population of dendritic cells or population of dendritic-like cells is a population of dendritic-like cells.

51. The method according to claim 50 wherein the dendritic-like cells are myeloid dendritic-like cells.

52. The method according to claim 51 wherein the myeloid dendritic-like cells are derived from myeloid dendritic cells.

53. The method according to claim 52 wherein the cells derived from myeloid dendritic cells are myeloid leukaemia-derived cells.

54. The method according to claim 53 wherein the myeloid leukaemia-derived cells are selected from the group consisting of KG-1, THP-1, U-937, HL-60, Monomac-6, AML-193 and MUTZ-3.

55. The method according to any one of the preceding claims wherein the dendritic-like cells are MUTZ-3 cells.

56. The method according to any one of the preceding claims wherein the one or more negative control agent provided in step (c) is selected from the group consisting of 1-butanol, 4-aminobenzoic acid, chlorobenzene, dimethyl formamide, ethyl vanillin, isopropanol, methyl salicylate, propylene glycol, potassium permanganate, Tween 80™ (polyoxyethylene (20) sorbitan monooleate) and zinc sulphate.

57. The method according to claim 56 wherein at least 2 control non-sensitizing agents are provided, for example, at least 3, 4, 5, 6, 7, 8, 9, 10 or at least 11 control non-sensitizing agents.

58. The method according to any one of the preceding claims wherein the one or more positive control agent provided in step (e) comprises or consists of one or more agent selected from the group consisting of ammonium hexachloroplatinate, ammonium persulfate, glutaraldehyde, hexamethylen diisocyanate, maleic anhydride, methylene diphenol diisocyanate, phtalic anhydride, toluendiisocyanate and trimellitic anhydride.

59. The method according to claim 58 wherein at least 2 control sensitizing agents are provided, for example, at least 3, 4, 5, 6, 7, 8, 9, 10, 11, 12, 13, 14, 15, 16, 17, 18, 19 or at least 20 control sensitizing agents.

60. The method according to any one of the preceding claims wherein the method is indicative of the sensitizing potency of the sample to be tested.

61. An array for use in a method according any one of the preceding claims, the array comprising one or more binding moieties as defined in any one of claims 19 to 29 and 31-38.

62. An array according to claim 61 wherein the binding moieties are capable of binding to all of the biomarkers defined in Table 1A.

63. An array according to claim 61 or 62 wherein the binding moieties are capable of binding to all of the biomarkers defined in Table 1B.

64. An array according to claim 61, 62 or 63 wherein the binding moieties are capable of binding to all of the biomarkers defined in Table 1C.

65. An array according to any one of claims 61 to 64 wherein the binding moieties are capable of binding to all of the biomarkers defined in Table 1.

66. An array according to any on of claims 61 to 64 wherein the binding moieties are immobilised.

67. Use of two or more biomarkers selected from the group defined in Table 1 in combination for identifying respiratory hypersensitivity response sensitising agents.

68. The use according to claim 67 wherein all of the biomarkers defined in Table 1 are used collectively for identifying hypersensitivity response sensitising agents.

69. An analytical kit for use in a method according any one of claims 1 to 60 comprising:
A) an array according to any one of claims 61 to 66; and
B) instructions for performing the method as defined in any one of claims 1 to 60 (optional).

70. An analytical kit according to claim 76 further comprising one or more control samples.

71. An analytical kit according to claim 69 comprising one or more non-sensitizing agent(s).

72. An analytical kit according to claim 69, 70 or 71 comprising one or more sensitizing agent(s).

73. A method or use substantially as described herein.

74. An array or kit substantially as described herein.
